(19) United States
(12) Patent Application Publication
(10) Pub. No.: US 2026/0174381 A1
(43) Pub. Date: Jun. 25, 2026

(54) Breast Sense Feeding Monitor

(71) Applicant: May & Meadow, Inc., Redwood City, CA (US)

(72) Inventors: Hooman Hafezi, Redwood City, CA (US); Mary Judith Insun Feezer, Redwood City, CA (US); Douglas Alan Webb, Los Altos, CA (US)

(21) Appl. No.: 19/462,170

(22) Filed: Jan. 28, 2026

Related U.S. Application Data
(63) Continuation-in-part of application No. 17/862,146, filed on Jul. 11, 2022, now abandoned.
Application No. 17/862,146 is a continuation-in-part of application No. 16/332,589, now abandoned.
(60) Provisional application No. 62/393,673, filed on Sep. 13, 2016.
(60) Provisional application No. 62/481,572, filed on Apr. 4, 2017.

(51) Int. Cl. A61B 5/00; A61B 5/0535

(52) U.S. Cl. CPC A61B 5/4288; A61B 5/0535; A61B 5/4312; A61B 5/6802; A61B 5/685; A61B 5/742

(57) ABSTRACT

The Breast Sense Feeding Monitor provides real-time measurement of breastfeeding metrics including milk volume and infant suck and swallow characteristics over multiple feedings in home and clinical settings. The system utilizes machine-learning to combine data from multiple sensors and provide accurate, personalized real-time data to support breastfeeding mothers.

### CROSS-REFERENCE TO RELATED APPLICATIONS

This application is a continuation-in-part of U.S. application Ser. No. 17/862,146 filed Jul. 11, 2022, which application is a continuation-in-part of U.S. application Ser. No. 16/332,589 filed Mar. 12, 2019, which application is a U.S. National Stage Application of PCT Application No. PCT/US2017/051419 filed Sep. 13, 2017, which application pursuant to 35 U.S.C. § 119 claims the benefit of U.S. Provisional Application Ser. No. 62/393,673 filed Sep. 13, 2016, and U.S. Provisional Application Ser. No. 62/481,572 filed Apr. 4, 2017, which applications are hereby incorporated by reference in their entirety.

### BACKGROUND

An infant's ability to feed successfully is critical to their development. For newborns, especially those born prematurely, the ability to assess feeding is often critical to the child's care.

The adage “breast is best” has gained prominence both with clinicians and in the community generally. Breast milk is known to be the ideal food for babies nutritionally and to avoid colic, a serious problem for some infants. Often the antibodies a mother conveys to her child through breastmilk protect the child from disease, or mitigate illness when it occurs. Breast feeding also helps the mother and child bond emotionally.

Successful breast feeding in developing countries it particularly critical to a baby's wellbeing. With limited medical care available, vulnerable newborns and infants often suffer tragically high fatality rates. Breast milk can mitigate this serous risk, both through ideal nutrition that is easily absorb by the baby, and protective antibodies. Also, breast feeding's hormonal effects on the mother naturally providing broader spacing in her pregnancies, even without contraception. This is an important factor in both maternal and child health.

Additionally, in developing countries, baby formula is relatively expensive and of limited availability. If formula is resorted to early in a child's development, it is unlikely they will return to breast feeding. Worse, because of the cost and lack of a reliable supply chain for baby formula, children in developing countries are often have limited access to or are denied even this less optimal source of critical nutrition

Thus, in both developing and developed countries, information that encourages and enables breast feeding is of prime importance to the health and wellbeing of babies. Specifically, concrete feedback that allows better breast feeding and assures the mother and father that their child is receiving adequate nutrition from breast milk can encourage and reinforce successful breast feeding.

While the amount of milk taken by babies can be readily determined with formula feeding, the amount of breast milk consumed by a baby is often an unknown quantity. Unfortunately, the concern that their baby may not be feeding enough at the breast for optimal growth often causes mothers to abandon breast feeding in favor of formula feeding. While this suboptimal nutritional source is a disadvantage to babies in developed countries, resorting to formula feeding in developing countries can have tragic consequences in the resulting morbidity and mortality of young babies.

Some basic approaches to determine how much breast milk a baby receives have included weighing a baby before and after a feeding, or weighing their diapers to determine how much fluid and solid matter has been taken in from the breast milk. However, these are cumbersome and inexact methods, and so are rarely used on an ongoing basis. For premature infants and newborns receiving colostrum from their mothers, these methods are not practically applicable to the small volume of nutrition being received.

Scientists have responded to these needs of babies, parents and clinicians for information on breast feeding by developing devices which can provide some insight into a baby's ability to effectively nurse and receive breast milk. By example, Gurtwein teaches the weighing of the mother's breast before and after feeding her baby to estimate how much milk the child received, U.S. Pat. No. 9,211,366 B l issued Dec. 15, 2015.

Larsson teaches a ridged breast shield that uses electric resistance measurements to estimate how much milk a mother produces and the baby ingests U.S. Patent Application 2005/008035 A1, published Apr. 14, 2005. Kapon et al teach a device to assess the volume of milk cells with capacitance measurements of the breast before and after feeding. U.S. Pat. No. 9,155,488 B2 issued Oct. 13, 2015.

Currently available breast milk assessment devices can provide some information on the milk production and child feeding, typically for a single point in time. Unfortunately, these readings often do not accurately reflect the overall nutrition being provided to the infant. Also, these devices are more suited to a clinical setting, and so cannot practically provide important information to parents when the baby comes home. Information on home feedings is particularly useful, as it reflects the day to day nutrition of the baby, and gives parents ongoing feedback that their child is nursing successfully.

With the advent of personal electronic devices, and the movement for personalized medicine, such devices as Fitbit have put some of the power of clinical tests into home use, to good effect. However, these capabilities have not yet been put into the hands of parents wanting to assess their baby's ability to feed from their mother's breast.

It would be an important advancement if calculation of breastmilk provided to a baby could be provided on an ongoing basis in real time, both in home and clinical settings. This innovation would be especially valuable if it provided biofeedback to coach mothers and lactation consultants on optimal nursing techniques.

### SUMMARY

The Breast Sense feeding monitor includes a wearable patch worn on a mother's breast and a mobile app that provides accurate, real time data on the volume of breast milk expressed from the breast and consumed by a nursing baby. This new system is an engineering breakthrough for meeting both the needs of parents and clinicians, as it can be used both in clinical and home settings. In developing countries, Breast Sense feeding monitor has the potential to assure better health of babies and save the lives of infants.

To accomplish these unique capabilities, the system relies on an impedance sensor circuit to detect changes in the breast's milk content based on changes in breast's electrical properties. The core innovation of the Breast Sense system is to combine impedance measurement of tissue fluid content with a shape measurement element to achieve an unprecedented level of accuracy, flexibility, comfort, and ease of use. This allows regular, daily measurement of breastfeeding milk volume and other metrics related to infant feeding.

The shape measurement element may be a strain gauge, such as a piezoelectric sensor, that is incorporated into the wearable patch. Alternatively, the shape sensing element may be a separate optical infrared device such as a cell phone camera, portable camera, or LIDAR sensor that generates images of a breast which are subsequently analyzed by software to measure breast shape through a technique known as photogrammetry. The shape sensing element substantially improves the accuracy and ease of use of the impedance sensor circuit to deliver a real time monitoring capabilities in both home and clinical settings. It is a long-needed tool for optimizing breast feeding outcomes.

### Ease of Use

The small, flexible form factor of the Breast Sense patch enables it to be applied comfortably and conformably to the breast of a breastfeeding mother. This important advancement allows comfortable wear for 12 hours or more, allowing multiple feedings to be measured continually and overtime. The resulting large and comprehensive data set provides a very accurate reading of the baby's feeding habits and capabilities.

Moreover, the simplicity of the Breast Sense feeding monitor design as compared to previously available systems allows the readings to be taken in the natural setting of a home feeding. This provides a more realistic determination of the baby's feeding patterns and the amount of milk the baby is receiving.

Ease of use and the ability to easily take measurements over multiple feedings sessions is very important for at home use by mothers and babies because an infant's feeding behavior, including appetite, changes substantially from feed to feed. Therefore, a highly precise but cumbersome measurement of feeding characteristics and milk intake in a single feeding is of low value, since variations in appetite and infant alertness can result in 2× or more difference in milk intake from feed to feed. Conversely, a wearable device that provides great ease of use over multiple feedings at the expense of some accuracy in a single measurement is ideal for these mothers and babies. Ease of use includes single handed and robust operation and zero or minimal effort required from a mother to maintain or calibrate the system.

The Breast Sense feeding monitor systems e-data capabilities also enable, for the first time, remote coaching by lactation specialists based on accurate and objective real-time data. It also enables automated app-based biofeedback and lactation coaching for mothers and provides pediatricians and nurse practitioners remote access to real-time data on babies' health and development.

### Flexible Sensing Patch

A configuration of the Breast Sense wearable device is a flexible patch that is attached to and conforms to the breast. As explained in more complete detail below, the fully integrated patch is provided with four or more impedance electrodes. In the basic version of the Breast Sense feeding monitor, the electrodes are provided linearly in pairs, with soft fabric in between the electrodes. However, there are more complex and nuanced shapes and configurations possible that may provide advantages in certain situations.

The patch is designed to be light and comfortable for a mother to wear for an extended time, up to 12 to 24 hours. A single button allows for controls such as wakeup, start and stop based on an unambiguous tap pattern.

The patch is also designed to balance comfort with functionality. Typically, the sensing patch has a form factor similar to a large BAND-AID® roughly 6 inches long. An even shorter version optimizes comfort. However, in some embodiments of the Breast Sense feeding monitor where sensitivity is critical, such as a clinical setting, the sensing patch can be larger and extend from the mother's sternum to her rib cage.

The Breast Sense patch may take on a variety of shapes to ensure accuracy and ease of placement on the breast. By example, the patch can be incorporated into a single linear or curved body. It may also be integrated a patch with 2 or more segments connected to one another via conductive components such as wires or a flexible circuit board.

### Shape Sensing Element

In embodiments where a strain gauge sensor is integrated into the patch, the data generated by the impedance sensor and the strain gauge are aligned or coordinated in time. This is accomplished by the analog signals from both sensors being fed to a suitable A/D converter and processor and converted into a single digital data stream before wirelessly transmitted to the mobile phone. This is important to avoid latency. Latency occurs when one or more a wireless signal is sent to a mobile phone that is handling multiple operations at the same time. When the signals arrive, there may be a delay or latency in processing the signal if the phone is in the middle of other operations.

In the Breast Sense feeding monitor, a shape sensor is used in concert with the impedance sensors to provide unprecedented capabilities to measure breast milk consumption by babies while ensuring convenience for mothers As described in more detail below, the shape sensor strain corrects for differences in breast size, shape, and curvature among different subjects and for changes in the breast size and shape over the course of weeks and months post-partum. In certain configurations, it may also detect and correct for distortions caused by movement in the breast due to breathing, laughing, or coughing or the strain baby sucking, squeezing or swatting at the breast. If left uncorrected, these factors can affect the accuracy of data.

### Impedance Sensing Circuit

Determination of milk quantity fed to an infant or milk flow rate during breastfeeding can be accomplished using a bio-impedance measurement, similar to that used for body fat content measurement. A decrease in the milk/fat ratio in the breast results in an increase of the electric impedance in the breast. An applied sinusoidal or square wave current (typically <1 mA) will produce a voltage detected by electrode on the breast. The voltage will provide a direct measure of the impedance change due to milk flow. Furthermore, the detected voltage signal will exhibit a phase characteristic of the amount of conductive (milk) to nonconductive (fat) matter. This is a similar principal to that used in bodyfat composition analyzers (e.g. the Omron HBF 306C system). Typical frequencies are in the range of 1 kHz to 300 kHz. Typically, 2 to 4 electrodes are applied to the breast in suitable locations. The electrodes may be similar to those for an EKG measurement (gel electrodes), applied to three locations around the breast, or to the breast and back of the mother. Alternatively, at least one of the electrodes may be a microneedle that penetrates the top skin layer. This configuration is attractive because it removes the contribution of galvanic skin conductance from the measurement. Electrode design

Breast sense feeding monitor can utilize a number of sense and drive electrode designs. Electrodes similar to rectangular or circular EKG style gel electrodes can be employed. However, in some situations it may be advantageous to use annular electrodes have advantages in capturing data on the entire tissue of the breast. Annular electrodes also allows multiple electrode mapping if there are multiple milk annuli. This feature is shown in more details, below.

Microneedles used as the interface between the electrode and the breast allows measurement beneath skin. This choice in electrode design can limit or eliminate electrode-skin resistance problems in testing.

Multi-electrodes provide better sensitivity in data collection than single electrodes. It is advantageous to select the electrode that gives largest change for capacitance. The system interpolate electrode readings to get highest change data, providing breast volume and mapping the breast.

The electrodes can sense at various frequencies. By example, they can sense at 1-300 kHz, specifically at 1-100 kHz, and most specifically at 5 to 50 KHz. Sampling data at a single frequency is simplest, and has the advantage of the lowest power consumption, but less reliable.

Other techniques to improve raw data based on various frequencies can be used to provide greater accuracy. By example, data can be taken at two frequencies, and if they agree, the data is confirmed. If they disagree, the measurement is repeated. Three or more frequencies can be tested. If two agree, that measurement is used; if they do not agree, the measurement is repeated. These approaches are typically automated in the system.

These problems of differences in breast size and shape among subjects or changes in breast size over time are remarkably ameliorated by the combined use of an impedance and a shape sensor while ensuring comfortable form factor that allows free movement of mother and baby, and so provides a much more natural feeding position. This advantage encourages long-term and regular use of the sensor which reflects the actual feeding habits of the baby than measurements taken less frequently.

Currently available breast feeding monitors, including those based on impedance measurement alone, have limited sensitivity because the electric signal detected by the impedance sensor is sensitive not only to milk content, but also to the shape and size of the breast. This is because the breast size, shape, curvature, and distortions change the distance between the impedance electrodes or the cross-sectional area of breast tissue available for current to flow, and can thus result in inaccurate measurements if ignored.

As a result, the impedance electrodes in some previous commercial systems such as the MilkSense™ system were affixed to a rigid support structure to ensure constant spacing and curvature relative to each other. Furthermore, the mother had to be motionless and in a consistent position in order to get consistent results. These prior restrictions cause significant inconvenience for mothers and babies, reduce sensitivity, and prevent effective measurement of milk transfer in real time.

### Calibration

An important innovation unique to the Breast Sense feeding monitor is eliminate the need for subject-specific calibration at the point of use. This allows a mother to use the Breast Sense feeding monitor without the need for a lengthy and laborious calibration procedure to determine calibration parameters that convert impedance signals to milk volumes with high accuracy for each mother and potentially each breast.

The Breast Sense system solves this problem by using its shape sensor to determine the breast shape with little or minimal effort from the mother. The parameters that characterize breast shape, such as breast curvature, are calculated directly from a built-in strain gauge sensor or by obtaining pictures of the breast using a cell phone camera and constructing a 3D model of the breast using a technique known as photogrammetry. These parameters are then used to lookup an appropriate calibration curve for each mother based on a set of calibration data measured ahead of time with a reference cohort of subjects. Combining the impedance sensor with measurement of breast shape allows the Breast Sense feeding monitor to eliminate individual feeding calibration and makes the system much more useable for home applications.

### Suck and Swallow Patterns

In addition to measurement of milk volume, real time measurement of infant suck patterns during breastfeeding is of interest to detect or potentially diagnose conditions that may affect in infant's ability to feed normally. Anatomical or neurological conditions that causes problems with swallowing, breathing, or sucking result in irregular or disorganized suck ore feeding patterns. Disorganized suck is common in babies with ankyloglossia (tongue-tie), Lip tie, High or narrow palate, Micrognathia/retrognathia (small or recessed jaw), Cleft lip or palate, and other conditions that affect latch. It may also occur with mothers with breast issues such as inverted nipples or other conditions that interfere with proper latching.

The Breast Sense feeding monitor can measure suck patterns by using its impedance sensor, which is sensitive to distortions in breast shape and can easily pick up suck patterns. In embodiments where the strain gauge sensor is present, the strain sensor can provide similar information.

Detection of suck patterns is also useful in estimation of colostrum volume, low milk volumes, or the progression in milk production immediately after birth and during the first 1-2 days after birth or for premature infants in the NICU. After birth, an infant's suckling movement promotes the production of hormones that initiate milk production. During the first 1-2 days after birth, the breast initially produces a small amount fluid known as colostrum. Colostrum has a thicker consistency and lower volume than the breast milk generated once milk production has fully commenced (past the onset of lactogenesis II). If the volume of colostrum is too low, it may be difficult to measure it with precision using changes in breast impedance alone. However, measurement and detection of a regular suck pattern and the so-called suck-to-swallow ratio can be used as an indicator of successful feeding and used to measure and track the gradual increase in milk production. Eventually, once the milk volume is sufficiently high, the impedance sensor may be utilized.

Another application for detecting sucks and swallows is to assess feeding ability in high-risk infants with medical conditions that can affect feeding ability. Infants with neurological problems, such as premature infants, often have difficulties in coordinating the suck-swallow-breath motions required for successful feeding. These infants will typically suck a few times, but are unable to sustain a succession of sucks for effective feeding. Furthermore, the number of sucks in between swallows can indicate the infant's suck strength, a useful metric for monitoring progress in infant's recovering from trauma, such as cardiovascular defects and surgery. Because feeding requires a great deal of exertion in these infants, monitoring their suck patterns during feeding is akin to monitoring a heart patient's heart rate while running on a treadmill and can provide a means of assessing their progression, strength or recovery from trauma.

In certain situations, a more precise measurement of an infant's sucks and swallows are desired than would be possible with a sensor located on the mother's breast. In these applications, a smaller “Baby Sense” patch can be placed on the infant's chin or throat. The intent is to detect movements that correspond to sucks and swallow and potentially breathing in a location on the infant's body that provides better sensitivity than a patch on the mother's breast.

This “Baby Sense” patch can be used in conjunction with the “Breast Sense” patch on the mother's breast. In some instances, the “Baby Sense” patch may also be used separately on its own. It would contain a strain gauge or an impedance sensor, or both.

### BRIEF DESCRIPTION OF THE DRAWINGS

FIG. 1 is a diagrammatic view of the Breast Sense Feeding Monitor system, showing the components in the first and second layers of the wearable patch.

FIG. 2A provides a broad view of the Breast Sense Feeding Monitor system in use by mother.

FIG. 2B shows a larger view wearable patch of the Breast Sense Feeding Monitor system.

FIG. 2C shows a larger, more detailed view of mobile phone and GUI of the Breast Sense Feeding Monitor system.

FIG. 3A shows the basic patch design for the Breast Sense Feeding Monitor system.

FIG. 3B illustrates a distributed patch design for the Breast Sense Feeding Monitor system.

FIG. 3C is a hybrid configuration where the sensing second layer is divided into two pieces.

FIG. 3D is a detailed view of FIG. 3C.

FIG. 4A shows a basic arrangement for the impedance sensing electrodes.

FIG. 4B shows a design with more than four impedance sensing electrodes.

FIG. 4C is a cross-section of typical gel electrode.

FIG. 4D illustrates a top view of an alternative electrode configuration.

FIG. 4E illustrates bottom view of the patch with alternate electrode configuration.

FIG. 4F illustrates a top view of the wearable patch with feature for reproducible positioning on the breast.

FIG. 4G shows a patch incorporating multiple electrode pairs for electrical impedance tomography

FIG. 5A illustrates the electrodes to be applied to the breast prior to measurement.

FIG. 5B shows what the patch after the measurement is complete.

FIG. 6 shows the microneedle electrode design.

FIG. 7A shows a strain gauge sensor bending measurement in one direction.

FIG. 7B shows two strain gauge sensor in different directions.

FIG. 8A is a graph of the output of an impedance sensor for a typical feeding session.

FIG. 8B is a graph of the strain gauge output.

FIG. 8C data from impedance and strain sensors and the combinations of the data to reduce noise.

FIG. 8D shows UNIVERSAL calibration curve.

FIG. 9A shows the operation of the Breast Sense Feeding Monitor system at different frequencies.

FIG. 9B shows showing detection of suck during the single frequency part of the measurement.

FIG. 10 shows a patch configured with an additional impedance or strain sensing location in the latch area to detect sucks and swallows.

FIG. 11A shows the simultaneous use of Breast Sense Patch and Baby Patch on the baby and breast.

FIG. 11B shows real time milk volume output from the Breast Sense Patch.

FIG. 11C shows detection of sucks and swallows at the additional sensing location.

FIG. 12A shows an example of a patch applied to the breast with individual calibration.

FIG. 12B shows an alternative approach is a patch with design features that provide alignment to both breast and breast pump flange.

FIG. 13A shows an example of data collected with the configuration described in FIG. 12A.

FIG. 13B shows impedance calibration data from multiple subjects collected with the configuration in FIG. 12A. This data is suitable for using subjects as long as their breast anatomy falls within the range of subjects used to generate the calibration curve.

FIG. 14. shows a work flow for using a camera and photogrammetry as shape sensing element along with a machine learning model to achieve more accurate calibration of the impedance sensor

FIG. 15. Shows the steps involved in using images from a cell phone camera to generate a 3D model of the breast and patch that serves as one of the inputs to the machine learning model

FIG. 16. Shows typical data used to train the machine learning model. This data is used to look up or compute accurate calibration parameters for the impedance sensor.

### DETAILED DESCRIPTION

### System Components

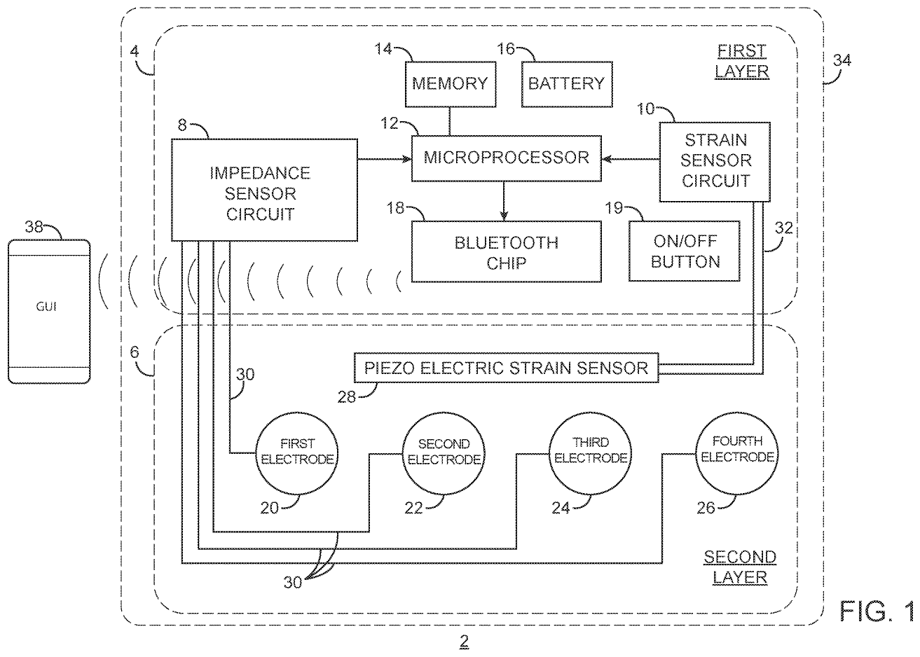

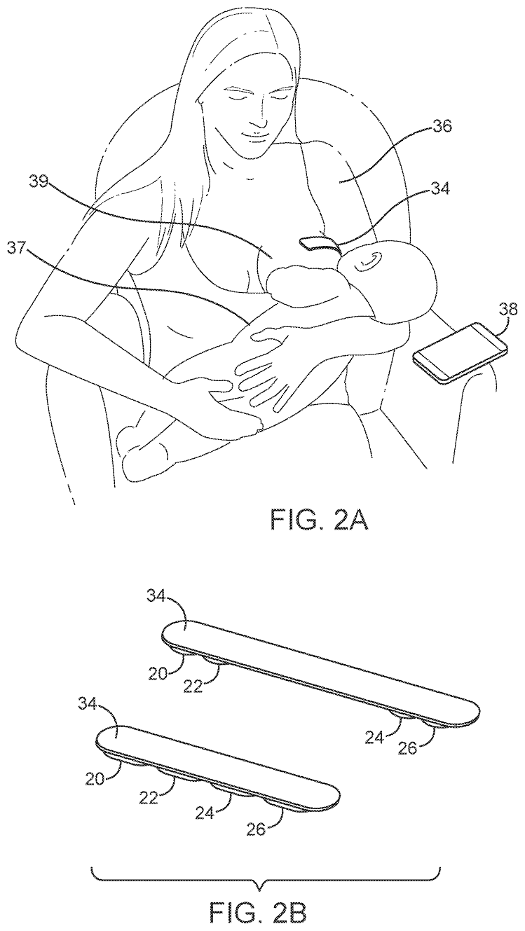

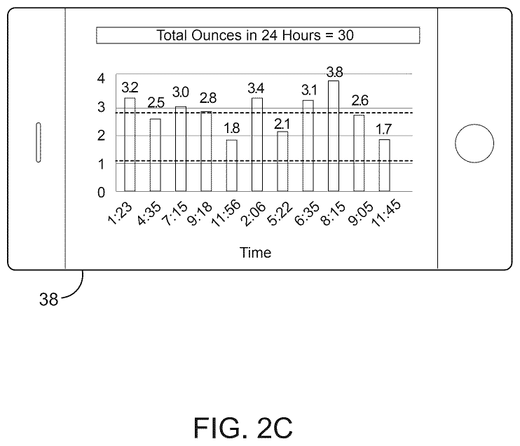

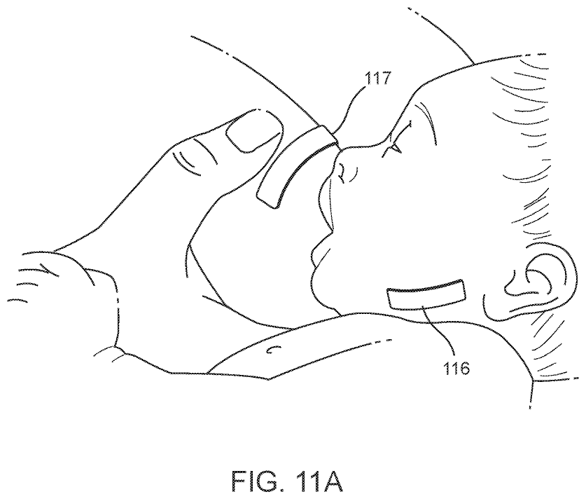

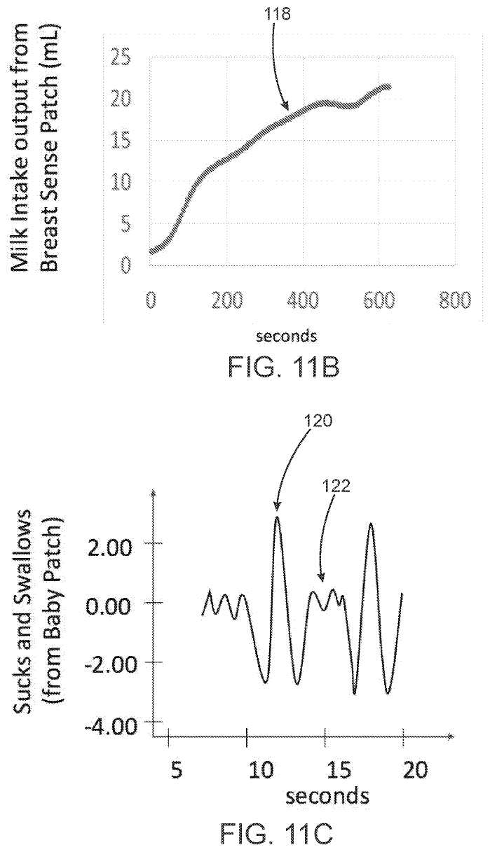

The Breast Sense Feeding Monitor system achieves its unique advantages and capabilities through the synergisms between its key components. A central feature of the Breast Sense Feeding Monitor system is a wearable electronic patch, the Breast Sense Patch. The Breast Sense Patch detects changes in the breast's milk content as well as key parameters related to an infant's suck and swallow pattern. This information is communicated wirelessly to a mobile phone or other user interface. While collecting data, the Breast Sense Patch is placed on the mother's breast during one or more breastfeeding sessions. FIG. 1 shows one configuration of the Breast Sense Patch and its internal components as part of the Breast Sense Feeding Monitor system. FIG. 2A shows the Breast Sense Patch 34 and mobile phone 34 during use by a breastfeeding mother and baby. An optional additional patch, the Baby Patch, can be placed on the infant to collect additional data in certain cases and is described later in FIG. 11.

The components of the Breast Sense Feeding Monitor system can be designed in a variety of configurations. These configurations are selected to best suit a particular application. One such configuration of the Breast Sense Feeding Monitor components is shown diagrammatically as several blocks in FIG. 1.

As shown in FIG. 1, the Breast Sense Feeding Monitor system 2 includes Breast Sense Patch 34 which is composed of two layers of the system's components. These components work together to provide real-time sensing and reporting of the amount and rate that a baby receives breast milk from their mother. In some cases, additional information is obtained by the Breast Sense Feeding Monitor system, such as the baby sucking characteristics such as strength, rate, and quality.

In FIG. 1, first layer 4 is a physical region of the Breast Sense Patch 34. First layer 4 provides and supports much of the functionality the Breast Sense Feeding Patch 34. The circuitry in first layer 4 supports the sensing, calculation and reporting functions of Breast Sense Feeding Monitor 2.

Some examples of the circuitry which can be included in first layer 4 of Breast Sense Feeding 34 includes impedance sensor circuit 8, an optional strain sensor circuit 10, and microprocessor 12. Impedance sensor circuit 8 functions to apply a sinusoidal electrical to body through 2 drive electrodes 20 and 26, as shown below, sense the resulting voltage on the body using 2 or more sense electrodes 22 and 24 as shown below, and convert the detected quantities to digital signals for processing. Typically, the impedance circuit provides voltage sufficient to drive a current of up to of up to 1 mA RMS such as about 100 μA to 500 μA, through the breast tissue, at a frequency ranging from about 0.1 to 1 MHz, such as about 1 to 100 kHz.

In one implementation, the impedance circuit applies a voltage suitable for driving the desired current through the body, measures the resulting current flow and the voltage at the sense electrodes simultaneously, then processes the data to derive the desired output and transmit this information to the microprocessor 12. An example of an impedance sensor circuit is the Texas Instrument AFE4300 system on a chip. Alternatively, a custom circuit can be designed around a network analyzer chip such as the Analog Devices 12 bit AD5933. Other circuit designs suitable to this application will be well known to one of ordinary skill in the art.

The strain sensor circuit 10 is an optional component designed to measure breast shape in real time. It receives data from a sensor such as a piezoelectric or capacitive or resistive strain gauge. The sensor output is typically detected using a half or quarter bridge circuit, converts the analog signal to a digital signal, and transmits this information to microprocessor 12. In one embodiment, the TI AFE4300 System on Chip integrates both impedance sensing and strain sensing circuits into one package and can be used for both functions. Alternatively, a custom strain sense circuit is designed using an appropriate bridge circuit and differential amplifier such as the AD8220.

The communication of the impedance sensor circuit 8 and strain sensor circuit 10 to the microprocessor 12 are shown in this view by arrows indicating the direction of flow of information. While in this view of Breast Sense Feeding Monitor 2 the communication between the impedance sensor circuit 8 and strain sensor circuit 10 to the microprocessor 12 is via wires, in alternative embodiments of the Breast Sense Feeding Monitor 2 system, all three components maybe integrated into a single micro-circuitry chip or flexible circuit board.

Also provided in first layer 4 is optional non-volatile flash memory chip 14 and battery 16. Memory chip 14 serves to store software and settings to operate the Breast Sense Patch 14 and retain software and settings when the system is powered down. Also, should there be an interruption in power or delay in transmitting the data to the mobile phone 38, the non-volatile memory can retain some or all the data collected by the Breast Sense patch as a backup.

It is useful if memory chip 14 has at least about 20 MB storage capacity and preferably at least about 40 MB storage capacity, and write speed of at least about 10 kHz. A variety of memory chips can fulfill this requirement, such as the Cypress Semiconductor S25FL256S or equivalent chips.

Battery 16 provides power to all components contained in Breast Sense Feeding Monitor 2. Byway of example, the battery may be a lithium ion battery capable of providing about 3 to 3.8 V voltage and a capacity of about 120 mAh to 350 mAh, such as about 150 to 220 mAh over a discharge time of about 3 to 24 hours, such as about 5 to 10 hours, and a current of up to about 40 mA. This capacity provides total usage for at least ten 30-minu feeding sessions over the course of a day. Battery 16 may be rechargeable or non-rechargeable. Examples of non-rechargeable batteries include CR2032, R2032, CR2330, BR2330 batteries. Examples of rechargeable batteries include RDJ3032 or RDJ2440 batteries. If a rechargeable battery is used, a suitable charging circuit must be included in the battery component 16.

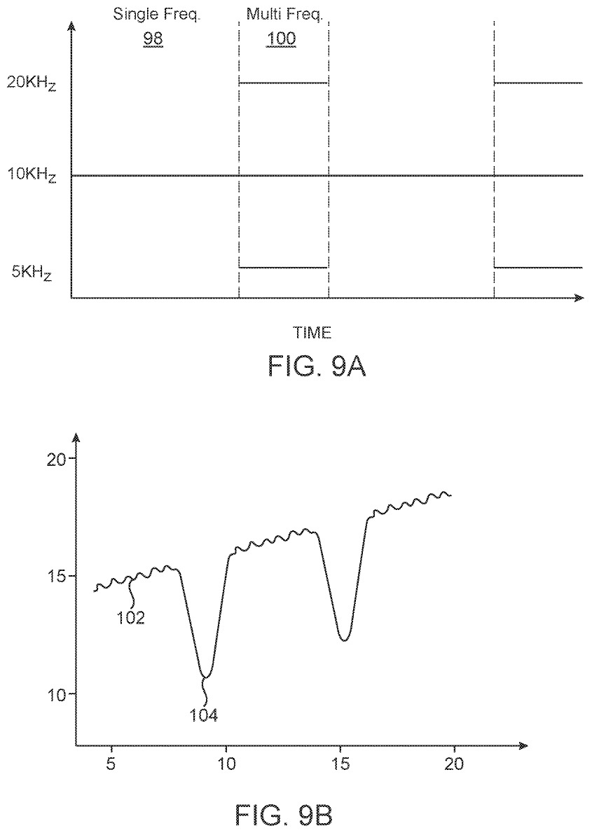

The battery component may further include power management circuitry to enable the Breast Sense Patch 34 to automatically enter a low power consumption “sleep” mode if no active feeding is occurring for a certain amount of time, such as about 2 or about 5 minutes. In sleep mode, the system may at least one sensor circuit at a low frequency to look for a signal characteristic of active feeding and “wake up” the Breast Sense Patch 34. An example of such a signal is the occurrence of high frequency, low amplitude undulations in the impedance sensor signal 102 or strain sensor signal 122 as shown later in FIG. 9B and FIG. 11C.

A Bluetooth chip 18 is provided for wireless transmission of data from the Breast Sense patch to cell phone 38. The Bluetooth chip 18 conveys key information, in a manner helpful and tailored to the user, to the cell phone 38 for communication to the user. In some embodiments of Breast Sense Feeding Monitor 2, some of the functions provided by the circuitry in first layer 4 is provided in said cell phone 38. In other embodiments of Breast Sense Feeding Monitor 2, the raw or partially processed data from the sensors in second layer 6 is transmitted to the cloud, processed, and then returned to the cell phone to be displayed to the user.

A variety of electronic components and combinations may be used to fulfill these functions. For example, the Cypress Semiconductor CYW20737 SOC and the Atmel ATBTLC1000 QFN BLE Bluetooth SoC incorporate microprocessor and Bluetooth chips into one component. The Silicon Labs EFR32BG1 chip is a microprocessor that provides at least about 20 MHZ clock speed and combines microprocessor, Bluetooth, program memory and ram, digital and analog i/o, real time clock, dc/dc converter, analog-to-digital and digital-to-analog converters, and Bluetooth into one package.

First layer 4 also contains optional on/off button 19. On/off button 19 allows the user, after applying the patch, to imply hit that button before breastfeeding, and then hit it again at the end of breastfeeding. This tells the device to go back into a sleep mode. A physical button has advantage over having this function controlled by the cell phone. For instance, during operation of the Breast Sense Feeding Monitor system, most mothers are handling a baby with one hand. Thus, in some cases, scrolling through screens and otherwise working on a cell phone is less convenient than having an actual button on the patch.

A physical on/off button provides that on and every time a measurement is to be accomplished, the user simply hits go, right, and the device runs. At the end of the measurement, the user hits the button again to turn the device and recording off. In a different embodiment, each time the button is hit, the device runs and collects data for the next half-hour. Within that button you can have sort of implements to make it robust. By example, a code can be implemented “two taps means start,” and “three taps means turn off.

Second layer 6 of Breast Sense Feeding Monitor 2 contains the impedance sensing electrodes for the impedance sensor circuit 8. The impedance sensing electrodes are first electrode 20, second electrode 22, third electrode three 24, and fourth electrode 26. In some configurations of the Breast Sense Feeding Monitor 2, there may be more or fewer of impedance sensing electrodes, but the minimum number of electrodes is 2. In many cases the preferred configuration is four electrodes, consisting of two electrodes to drive a sinusoidal current through the breast and 2 electrodes to measure the voltage.

The impedance sensing electrodes, first electrode 20, second electrode 22, third electrode 24, and forth electrode 26, are connect via connecting wires 30 to impedance sensor circuit 8. As described above, the impedance data for Breast Sense Feeding Monitor 2 from the impedance sensing electrodes and the optional strain sensor circuit 10 is conveyed to the microprocessor 12 and therein to the user.

Similarly, the optional strain sensor 28, also provided in second layer 6, connects via wire 32 to the optional strain sensor circuit 10. For the purposes of this application, strain sensor is defined as any mechanical sensor capable of detecting a deflection, or displacement of all or part of the Breast Sense Patch, such as a piezoelectric strain gauge sensor, capacitive mesh sensor, a pressure sensor, or equivalent. That information is then combined with the strain sensor data in the microprocessor 12 to provide more compressive, synergistic data to the user than that from the impedance sensing electrodes alone.

The sensor detecting breast shape may also be an optical sensor as discussed in greater detail elsewhere in this application. An example of this is a camera, such as the camera present in a mobile phone, that captures photographic images or videos of the breast from one or more angle. This method is sometimes referred to as photogrammetry. Software is used to process the images and build a 3-dimensional model of the breast that allows one or more breast dimensions to be measured and used to correct the impedance signal. Furthermore, markers may be applied to the breast using a pen, sticker, or temporary tattoo that serve as registration marks and allow the software to process images more accurately, more quickly, or more efficiently. Photogrammetry may be particularly effective at correcting for differences between different subjects or changes in breast shape for the same subject over the course of multiple days or weeks.

FIG. 2A provides a broad view of the Breast Sense Feeding Monitor 2 being used by mother 36. Mother 36 applies test patch 34, which contains all the components shown in FIG. 1, above, to her breast 39 before the first feeding of baby 37. In a typical Breast Sense Feeding Monitor 2 testing situation, mother 36 leaves test patch 34 on during the entire period during and between four or more feedings. The Breast Sense Feeding Monitor 2 uses its sensors to get a highly accurate measure of milk intake by the infant. In many cases, a number of other parameters are included in the analysis of the data. This final information is then transmitted those to mobile phone 38.

FIG. 2B shows a larger view wearable patch 34 in two different possible configurations among a range of different designs appropriate to the Breast Sense Feeding Monitor 2. The hardware of the Breast Sense Patch 34 can, in one embodiment, be designed and fabricated such that first layer 4 and second layer 6 are superimposed, so that they configured top of each other inside a wearable patch cover. The Breast Sense Patch 34 is from about 3-10″ in length, more specifically about 6″-8″ in length, and most specifically about 7″ in length. The Breast Sense Patch 34 is designed to be as thin and light as possible and may have a thickness of 2 to 35 mm at its thickest point, more specifically 2 to 15 mm, and most specifically about 10 mm.

FIG. 2C shows a larger, more detailed view of mobile phone 38. One approach to the graphic user interface is shown on the screen of mobile phone 38. However, the information can be conveyed to the mother 36 audibly as well, such as with a varying tone, or specific beeps when different points in the conveyance of the milk to the baby 37 are reached. Also, the information can be conveyed to a lactation specialist or other health care provider.

Note that while the device to provide information to the user is illustrated in this and the following figures as a smart phone, the interface can be any number of personal electronic devices, such as tablets, computers, TV screens, etc. Additionally, the user interface need not be graphic. By example, a speaker can provide audio cues, and vibration cues could also be employed.

In most applications, ease of use and comfort of the mother are far more important than the accuracy or precision of a single measurement. This is because an infant's appetite or milk intake can vary by more than a factor of 2 between feedings. Therefore, it is essential to take measurements over multiple feedings, typically about 4 to 6, to obtain a truly representative measure of an infant's feeding. Therefore, a patch that is more comfortable and can be readily worn over multiple feedings is preferable to a bulkier, less comfortable patch that may provide greater sensitivity for a single feeding session but is inconvenient to wear over multiple feedings.

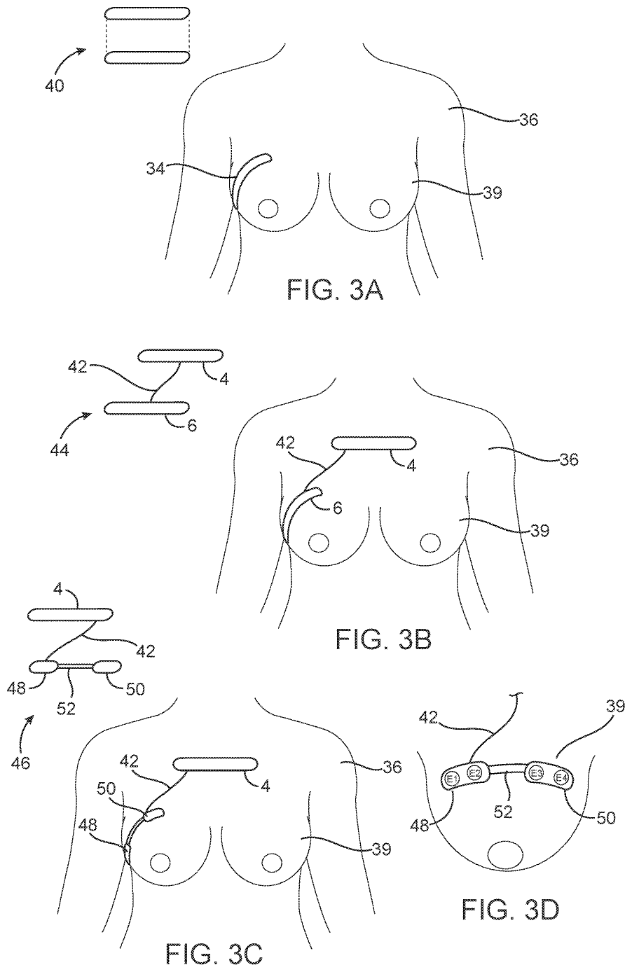

The Breast Sense Feeding Monitor system is highly adaptable to different form factors and applications, and can be designed in various configurations most suitable to a particular use. By example, clinical applications in a hospital will benefit from different designs than those used in a more consumer product application. FIGS. 3A, 3B, 3C, and 3D show alternative embodiments of the Breast Sense Feeding Monitor system 2 hardware configurations to provide different combinations of comfort and sensitivity.

In some applications, the configurations shown in FIGS. 3B, 3C, and 3D will have advantage over the basic configuration shown in FIG. 2B, exemplified in FIG. 3A. The configuration in FIG. 2B is basically two layers positioned directly on top of each other, combined into one body. In FIG. 3A, this two layer configuration 40 shows as a simple graphic the configuration described in greater internal detail in FIG. 1.

An alternative configuration as shown in FIG. 3B provides that the two layers of Breast Sense Feeding Monitor, first layer 4 and second layer 6 are configured as two separate pieces that are connected with layer connecting wire 42 to construct distributed patch design 44. In this case, first layer 4 of distributed patch design 44 can be positioned on the chest of mother 36.

Unlike the basic configuration shown in FIG. 1 and FIG. 2B, in FIG. 3B first layer 4 is its own packaged unit. First layer 4 contains the bulkiest electronic components, including the system battery. In this case, first layer 4 can, in certain embodiments of the Breast Sense Feeding Monitor system, be positioned on the mother's chest or elsewhere. Second layer 6 is separate from first layer 4. Second layer 6 contains only passive components such as electrodes and strain sensor and can be made extremely thin, flexible, and lightweight. The two are connected through layer connecting wire 42. Connecting wire 42 is basically a combination of connecting wires 30 into a wire bundle. In design 44, the light and highly conformal second layer 6 remains on the breast for multiple feedings. The bulkier layer 4 can be worn on the chest, sternum, armband or alternative location that is more comfortable or discreet. Alternatively, in additional embodiment, layer 4 can be located off the body, such as on a shelf or countertop. Layer connecting wire 42 would be a detachable wire that allows layer 4 and layer 6 to be connected when the mother and baby are about to commence a breastfeeding session and disconnected when the feeding sessions are over.

FIG. 3C shows a hybrid configuration 46 second layer 6 is divided or split into two pieces. This configuration provides even greater flexibility than the configuration shown in FIG. 3B. As a result, hybrid configuration 46 gives even more comfort in wearability of mother 36.

As in FIG. 3B, first layer 4 and second layer 6 are configured as two separate pieces that are connected with layer connecting wire 42. However, in this case second layer 6 is split into second layer part A 48 and second layer part B 50. Note that the layer connecting wire 42 can be connected to either second layer part A 48 or second layer part B 50.

In this embodiment, second layer part A 48 and second layer part B 50 each contain two electrodes. Second layer part A 48 houses impedance sensing electrodes, first electrode 20, second electrode 22. Second layer part B 50 houses impedance sensing electrodes third electrode 24, and forth electrode 26. These impedance sensing electrodes are not shown in this view.

As shown in both FIG. 3C and in further detail in FIG. 3D, second layer part A 48 and second layer part B 50 are both attached and spaced apart by a wire, flexible circuit board, or strain sensor layer 52 which contains the strain sensor 28. Thus, both the Impedance sensing functions and optional strain sensor may be incorporated in this distributed embodiment of second layer 6 in sensor layer 52. Note that layer connecting wire 42 can be attached to either second layer part A 48 as shown in FIG. 3C or second layer part B 50 as shown in FIG. 3D

Incorporating these design elements providing additional flexibility into an optimal Breast Sense Feeding Monitor system configuration provides long-term wearability for mother 36. The distinction between the separate configuration of the layers in the hybrid configuration is simply that second layer 6 is now split into separate units. Instead of this functionality being in one piece, there is a functionally longer piece. These two design configurations are not distinct in terms of function, only in physical configuration. It is more how the elements are arranged inside the housing, because these elements are still connected in terms of communication. The difference is the amount of stiffness that is ameliorated when the bulk housing is separated out.

The hardware configuration of Breast Sense Feeding Monitor can usefully be geared toward providing maximum flexibility, and resulting increased comfort, for mother 36. This advancement allows the measurement to be done for an extended duration, giving the most complete and accurate results. With this new functionality, the measurement is not actually a single measurement of a feeding, with maximum accuracy. Rather, the Breast Sense Feeding Monitor device 2 lends itself to measuring an average of total four or more connecting feedings. That insight is the motivation for these engineering features.

The total length and overall functionality of second layer 6 or second layer part A 48 and second layer part B 50, combined, in any of these configuration is important to achieving the best possible functionality for Breast Sense Feeding Monitor. As described previously, the length of the patch of second layer 6 is often 4-8 inches.

Some of the present inventors have developed data during studies of Breast Sense Feeding Monitor system prototypes around the effect of the length of second layer 6. This length influences signal strength, and so needs to be selected appropriately. The location of the patch containing second layer 6 on the breast is important. By example, it was determined that that placing the patch 3-6 centimeters from the nipple gives the best signal strength. This appears to be true in subjects with a range breast size and shape.

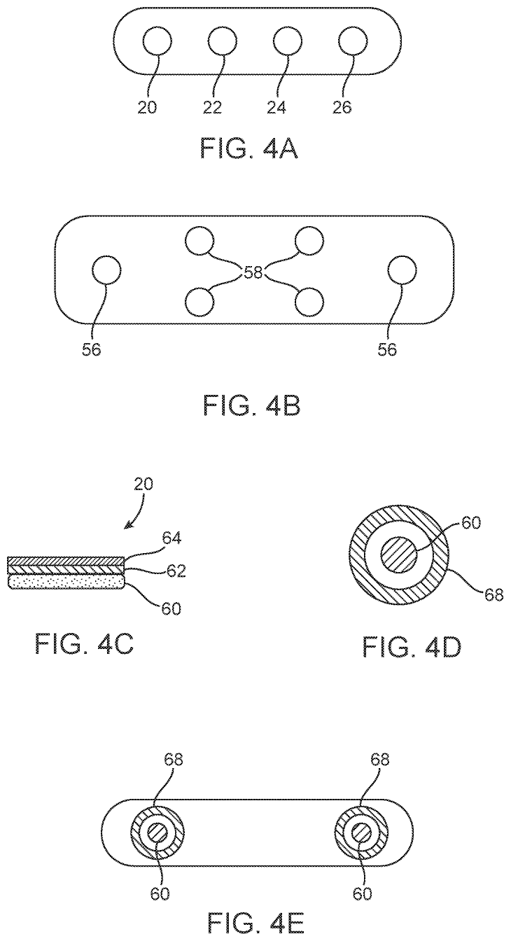

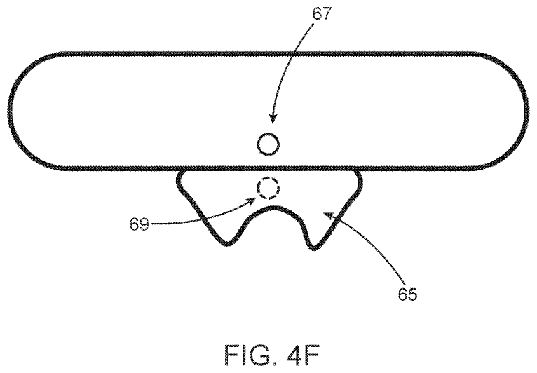

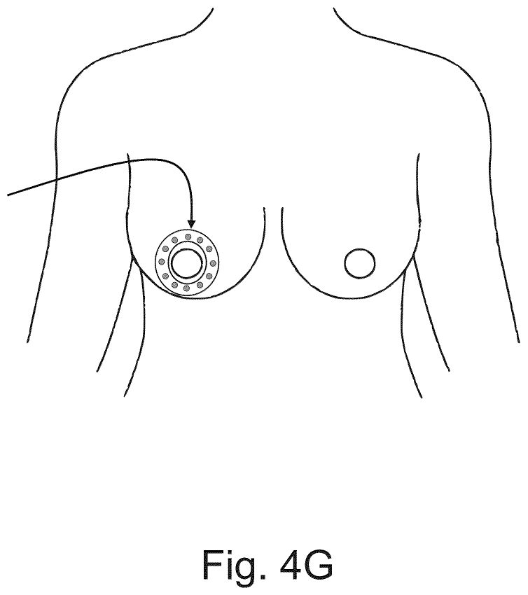

Because of the ease and flexibility of the Breast Sense Feeding Monitor system, mothers will be able to modify the placement of the sensor patch to the optimal location, both to optimize sensing and comfort, on breast 39. FIG. 4 shows different arrangements for electrodes for the impedance sensor, and various designs for the electrodes themselves. The most basic arrangement for the impedance sensing electrodes is shown in FIG. 4A. The impedance sensing electrodes, first electrode 20, second electrode 22, third electrode 24, and fourth electrode 26 are positioned collinearly, so that they are situated in a row within second layer 6. Two of them, first electrode 20 and fourth electrode 26, are conventionally considered the drive electrodes. These are the electrodes that are used to inject current into the body. The other two, second electrode 22 and forth electrode 24, are called the sense electrodes.

The difference between the sense electrodes and the drive electrodes is during the impedance measurement part of the device. The impedance sensor functionality involves driving a sinusoidal current through the drive electrodes in contact with the body. Then the voltage that exists in the body is measured by the Breast Sense Feeding Monitor system 2 with the sense electrodes.

Typically in impedance, two electrodes are used to inject current. Two other electrodes are used do the measurement. This is a convention, rather than a necessarily dedicated use of an electrode. Thus, the drive electrodes could be used to do sensing as well.

Multifunctional electrodes allow a flexible use of the Breast Sense Feeding Monitor system 2. By example, the Breast Sense Feeding Monitor system 2 can alternate between driving through first electrode 20 and fourth electrode 26, and sensing with second electrode 22 and third electrode 24. This mode of operation is in contrast to driving through electrode first electrode 20 and fourth electrode 26, and actually sensing with those same electrodes. Most typically, the Breast Sense Feeding Monitor system 2 will be driving with first electrode 20 and fourth electrode 26, and sensing with second electrode 22 and third electrode 24. The system can move fluidly between any of these modes, even very rapidly in the same session, to produce optimal functionality for the Breast Sense Feeding Monitor system 2.

FIG. 4B illustrates an alternative arrangement impedance sensing electrodes employing more than four electrodes. In this case there are six rather than four electrodes. In this embodiment of the Breast Sense Feeding Monitor system 2, drive electrodes 56 and sense electrodes as 58 are provided. Thus, in this configuration there are two more sense electrodes than show in FIG. 4A. During sensing, the drive current is still driven through electrodes 56. However, in this case it is possible to sense between different pairs of electrodes among the sense electrodes 58.

The advantage to this electrode configuration is that a potentially more accurate assessment of milk volume in the breast 39 can be provided. The milk reservoir in the breast, that is where the milk is stored in the breast, can be in different locations. This may not directly correspond to where the Breast Sense Feeding Monitor patch is applied to breast 39.

Because of natural anatomic variability, cells containing the milk may be higher or lower on different subjects. The greatest signal strength is if the sense electrodes are closest to where most of the milk reservoir are located. With multiple electrodes, there is the option of sensing different combination of electrodes and picking the one that gives the most signal.

This opportunity for optimal spacing this advancement represents is not currently available with existing systems. Appreciating and accounting for the effects of nodular pooling provides the opportunity to achieve fully accurate sensing data. For this reason, a configuration such as the one above is especially useful in a clinical setting, where highly accurate data in fewer test sessions is more important.

There are a variety of factors which can influence the optimization of data collection with the Breast Sense Feeding Monitor system. The breast changes over the course of feeding, both within feedings and over time. By example one pair of electrodes is more sensitive during the first few days of feeding after birth. Over time, the breast essentially maps itself out with the baby's changing in feeding and the changing milk consistency, content and volume. A different location for sensors may be optimal, say at week 2, 3, or 4 post-partum.

With these changes, having the multiple electrodes allows the Breast Sense Feeding Monitor system to be able to better handle and adjust to those changes, rather than simply rely on a minimum of four electrodes.

This heightened sensitivity and high accuracy will not be necessary for all applications. In some uses of the Breast Sense Feeding Monitor system 2, it may not be optimal to complicate the system, since this complexity can come with disadvantages of their own. For instance, the Breast Sense Feeding Monitor device would be bigger and, likely, less comfortable. Whether a design using just four electrodes, or one employing more is better suited to an application, will depend on the demands of the particular application and how truly accurate the results are required. In practice, some of the present inventors have found that four electrodes provide enough sensitivity for most applications.

Another advantage to having multiple electrodes in the Breast Sense Feeding Monitor system 2 is that the system can ‘sense’ through different pairs of electrodes in the 58 group, and generally map the location of optimal sensitivity by interpolating the signal. In this manner the signal can be assessed at various locations. With sensing from different pairs of electrodes map, the less sensitive spots could be identified, narrowing down to the most sensitive spot. For instance, the most sensitive spot may be located three quarters of the way between two different pairs of electrodes. To facilitate this mapping capability, more than four electrodes can be provided under 58.

This optional mapping function allows the potential for optimization of sensing, which is particularly key in applications such as clinical settings for preterm babies, or newborns receiving colostrum from their mothers. An embodiment of the Breast Sense Patch with a very high degree of mapping capability is shown in FIG. 4G. In this embodiment, the patch is circular and is configured to place a multiplicity of electrodes, such as 8 or more electrodes, around the nipple in a circular pattern. This configuration allows the fluid content inside the breast tissue to be mapped applying electrical excitation currents through a plurality of electrodes arranged on an external surface of the breast and measuring corresponding voltage responses at one or more additional electrodes. This method is known as electrical impedance tomography (EIT). The measured impedance data are processed to reconstruct a spatial distribution of electrical conductivity or impedance within the breast volume, thereby forming a map indicative of internal fluid content. Tomographic reconstruction may be performed using algorithms including, but not limited to, linearized back-projection methods, finite-element-based forward or inverse solvers, Gauss-Newton or Newton-Raphson iterative reconstruction techniques, and regularized optimization approaches such as Tikhonov regularization, total-variation regularization, or sparsity-constrained solvers. Impedance tomography has been used in applications such as imaging breast tumors or air gaps inside lungs based on differences in conductivity or permittivity between tumor and normal tissue and air and human tissue respectively. It can be used in breastfeeding to locate and track the distribution and amount of milk within a subject's breast which may be assessed from the intensity of the images. Alternatively, EIT may be used alone or in combination with a photogrammetric breast shape sensor techniques described in later paragraphs to establish the primary location of milk storing glandular tissue within the breast. Once established, the location of the glandular tissue can be used as in put to a machine learning model to select or compute an optimal CALIBRATION curve as described in detail below.

An important feature for the Breast Sense Feeding Monitor system 2 when used as a consumer product is its ease of use. In a home setting, the mapping system would be optional, and in many cases unnecessary to get key information. Some of the present inventors have been told by clinicians that it is preferable to have a simple, easy-to-use system for home use. However, in a doctor's office, a more full featured system with better resolution for this relatively shorter testing period would be more appropriate.

As shown in cross-section FIG. 4C, an electrode being used in the Breast Sense Feeding Monitor system as impedance sensors is typically gel electrode 20. Gel electrode 20 is provided with gel layer 60, silver-silver-chloride layer 62 and conductive backing 64. Examples of commercially available electrode types that may be suitable are 3M 2228, Vermed A 10022, and Covidien Kendall H69P neonatal gel electrodes. These gel electrodes serve to make electrical contact with the body and hold the Breast Sense Patch 34 in place. Custom made gel electrodes can be made with the desired size, shape, and adhesive strength to ensure comfort.

FIG. 4D illustrates a top view of an alternative electrode configuration. This alternative electrode design allows the drive and sense electrodes to be combined in a way that is more compact and gives better resolution. Again, there is a conductive section with a black silver-silver-chloride coating. A gel layer is provided to facilitate adhesion and conductivity.

However, in this case, a sense electrode 60 is at the center of the alternative electrode design. Sense electrode 60 has all the same layers as illustrated in FIG. 4C. But in this alternative electrode configuration, the sense electrode 66 surrounded by an annular drive electrode 68.

This kind of configuration had advantages over the basic electrode configuration illustrated in FIG. 4A. In FIG. 4A, there are four distinct electrode areas. The configuration in FIG. 4D allows the combination of these two and two, so you would effectively have two electrode areas.

This opportunity for different second layer 34 design is made possible by the FIG. 4C electrode is show in in FIG. 4E. The patch with the alternative electrode design would look more like a two-electrode patch. However, functionally, this is equivalent to the basic four electrode patch since each electrode area has functionally two electrodes.

One advantage of the electrode configuration in FIG. 4E versus that in FIG. 4A is that because there are only two electrode areas, the whole patch can be smaller and more flexible. With four areas, the patch can be a little stiff. However, by reducing the four electrode areas to two, there is more flexibility.

Another advantage of the electrode configuration in FIG. 4E versus that in FIG. 4A is in sensing. In the case of the electrode configuration in FIG. 4E, both sense electrodes 60 and drive electrodes 68 are provided.

In FIG. 4A the drive electrodes, first electrode 20 and fourth electrode 26, are outside the sense electrodes, second electrode 22 and fourth electrode 24. In 4E the sense electrodes 60 are measuring a voltage that is essentially right in the middle of the drive electrodes 68. This provides a bigger signal.

In FIG. 4A the biggest voltage difference is between the points at first electrode 20 and fourth electrode 26. When the sense electrodes are inside these points, only about half of that voltage is sensed. The voltage varying almost linearly from the points at 20 to 26, so only a portion of that is sensed. By contrast, the configuration shown in FIG. 4E results in a much larger signal, which is less prone to noise. This improves the signal-to-noise ratio is improved.

This configuration is also less sensitive to the exact location of where the milk reservoir is located. What happens is, if in FIG. 4A, the milk reservoir is right underneath this drive electrode, the measurement is accurate. However, if the sensing electrode is off to the side of the milk reservoir, the system will not catch the effect of that milk reservoir as well. Reduced signal strength results.

However, when the sensing and driving electrodes are collocated, as in FIG. 4E, there is a more generalized sensing. This is because whatever happens between, as the current travels through the breast is going to show up in these two sense electrodes. They cannot possibly be dislocated relative to the milk reservoir. The ideal situation is when those two are actually at the same location relative to the milk reservoir. That gives the biggest signal. The configuration shown in FIG. 4E can be modified to have multiple electrode areas, similar to FIG. 4B.

In summary, the various design strategies shown in configurations shown in FIG. 4A, FIG. 4B, FIG. 4C, FIG. 4D, and FIG. 4E can be generalized to the following design consideration. It is always possible add more electrodes to the Breast Sense Feeding Monitor system. However, an increased number of electrodes, while increasing sensitivity, comes at the expense of larger size and less comfort. Thus, the ordinary skilled artisan will consider design parameters for the end use to optimize effects and balance these considerations.

The Breast Sense Patch 34 may include an optional feature to enable consistent positioning of the patch relative to the nipple. FIG. 4F is a top view of a Breast Sense patch illustrating this feature. Feature 65 is a positioning flap made of fabric or plastic with a cutout for the nipple to allow the Breast Sense Patch 34 to be positioned at a precise distance from the nipple. Once the Breast Sense Patch has been positioned and attached to the breast, the flap is folded back onto the patch so that it is not interfering with the baby's latch. This is done using attachment components 67 and 69 which may be snaps, buttons, velcro patches, or other attachment mechanism.

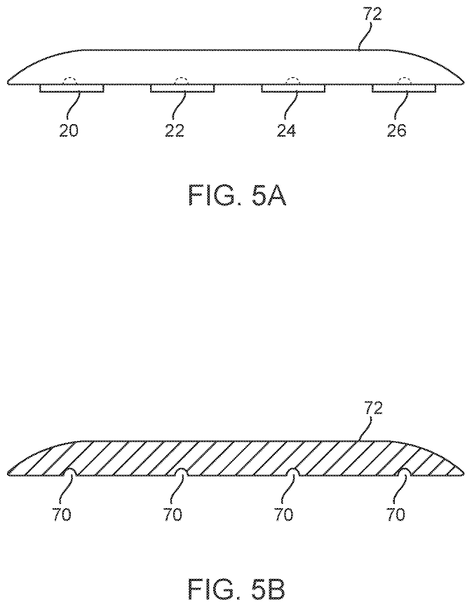

FIG. 5 illustrates that, when electrodes are described in the above figures, they are referring to removable electrodes. These are gel electrodes that adhere to the body. These gel electrodes snap into second layer 6 Thus second layer 6 is provided with little contact buttons for each electrode.

FIG. 5A is side view of FIG. 4A. FIG. 5A is 3-dimensional showing of how the electrodes 20, 22, 24 and 26 are set into the patch body 72. The electrodes as snapped-in and are, in some embodiments, removable. This removal can occur between uses of the Breast Sense Feeding Monitor system, or when the measurement is finished. FIG. 5A is an illustration with the electrodes in place is what is applied to the breast prior to measurements being taken.

FIG. 5B shows what the patch looks like after the measurement is complete. The user would remove the patch body 72, throw away the disposable part of the electrode. For the next measurement, the user would attach a set of four new electrodes 20, 22, 24 and 26 to snaps 70. Snaps 70 that allow the securing of the disposable electrode into the patch body 72.

There are many variants on this design which will be apparent to an ordinary skilled artisan. By example, the four electrodes can be provided on one backing piece. This makes it easy to put them on.

An adhesive gel electrode, which is shown in FIG. 4C, is the traditional way of making contact to the body for impedance, EKG, or other electrical measurements. Typically gel electrodes provide a certain amount of impedance or resistance to current flow. This is in addition to the impedance provided by the skin and breast tissue. In order to perform a measurement, the impedance sensor circuit 8 and battery 16 must provide sufficient voltage and power to drive the desired current, typically between about 100 and 500 uA. The injected current must also be applied at a sufficiently high frequency, typically greater than about 10 kHz, so that a substantial part of the current can reach the interior of the breast through capacitive coupling.

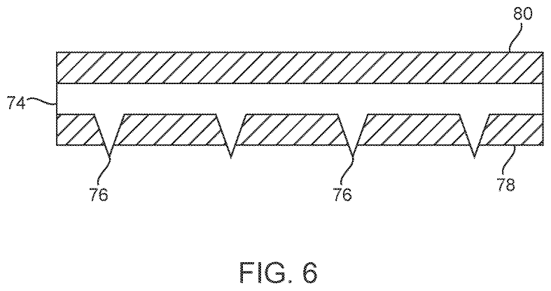

FIG. 6. illustrates employing a microneedle electrode 74 instead of a gel electrode in the Breast Sense Feeding Monitor system to optimize data collection.

In an alternative embodiment of the Breast Sense Feeding Monitor system shown in FIG. 6, the microneedle electrode 74 design is one where instead of a gel electrode, a microneedle electrode is used. The microneedle electrode has short, microneedles 76 that penetrates the skin slightly. Because the outer layer of the skin is very high resistance, better data is then obtained.

Microneedle electrode 74 has multiple microneedles 76, so there might be more than one. Microneedles 76 may be anywhere from 50 to 300 microns long. Microneedles 76 are typically made of stainless steel or silicon. Microneedle electrode 74 will still have adhesive layer 78 that would go either in between, or around the whole electrode. This adhesive layer 78 allows the microneedle electrode 74 to be applied and adhere to the skin. Also provided is conductive backing 80 for attaching a wire.

Microneedle electrode 74 may offer the advantage that they have a much lower resistance than the traditional electrodes. Microneedle electrode 74 multiple microneedles 76 are not deep enough to hit sub-dermal nerves. While thus not painful, microneedle electrode 74 may feel something like sandpaper to the user. As such microneedle electrodes 74 can have the disadvantage of being a bit uncomfortable to some users. However, this could be a good alternative for people who have a sensitivity to the adhesive.

Because the microneedle electrode 74 multiple microneedles 76 penetrate the dead layer of skin, they allow the current to be injected past that dead layer of skin. This means that the overall resistance to that current that is being injected by the drive electrodes is lowered. As a result, lower power is required, and a concomitantly smaller battery 16.

The battery 16 is a big part of the size of the patch. Employing microelectrode 74 in the design of the Breast Sense Feeding Monitor system can make the whole Breast Sense Feeding Monitor system device smaller and more comfortable to wear. This would be at the potential expense of local skin irritation.

Regarding the sense electrodes, because there is this dead layer of skin, the sense electrodes pick up the signal using “capacitive coupling”. The sense electrodes need to sense at several kilohertz to pick up that signal with the basic electrodes.

However, since electrodes with microneedles penetrate the dead skin, they actually make contact with the interstitial fluid just beneath that dead skin. As a result, the system can drive and sense at lower frequency, because the connection is ohmic. This is similar to the difference between connecting through a resistor versus through a capacitor. If the connection is through a capacitor, drive much happen at a high frequency. Thus use of microelectrode 74 has the potential to make the Breast Sense Feeding Monitor system circuits simpler, and lower power. This, in turn, would allow a smaller form factor for the Breast Sense Feeding Monitor system device.

### Use of Impedance Sensor Data to Measure Milk Volume

The primary means by which the Breast Sense feeding monitor detects milk volume during breastfeeding is by using impedance to detect changes in the electrical properties of the breast tissue during feeding. Because tissue with a high degree of fluid possesses different conductivity and permittivity than tissue with low fluid content, measurement of impedance at a suitable frequency allows determination of the milk volume present or removed from the breast. This approach is akin to widely available commercial devices that use bioimpedance sensors to detect hydration and body fat content.

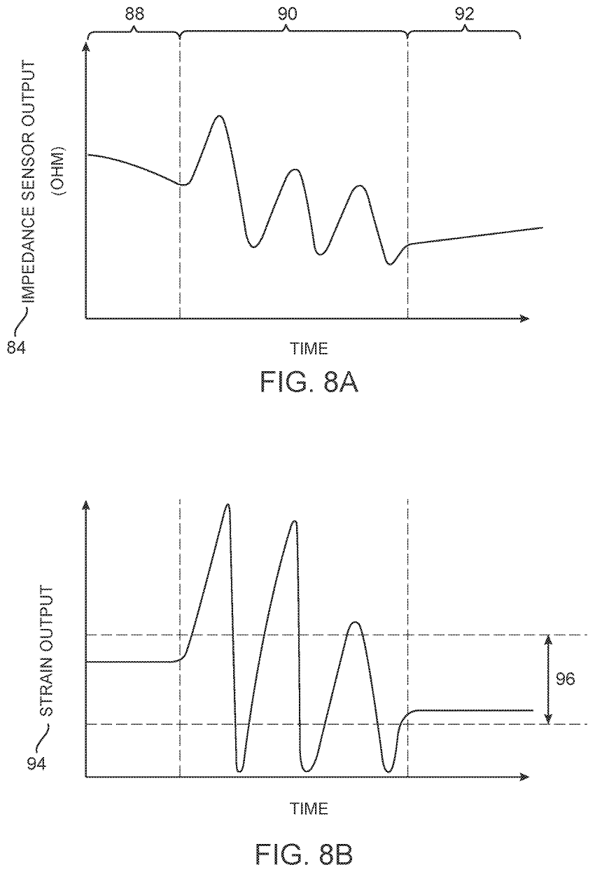

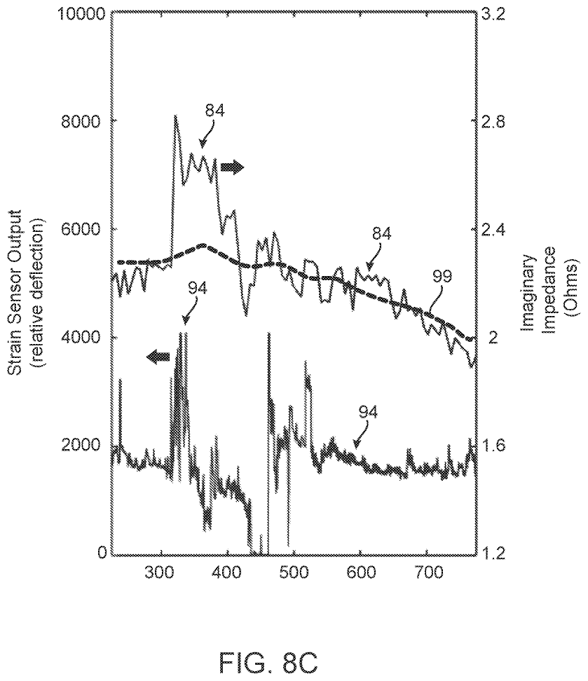

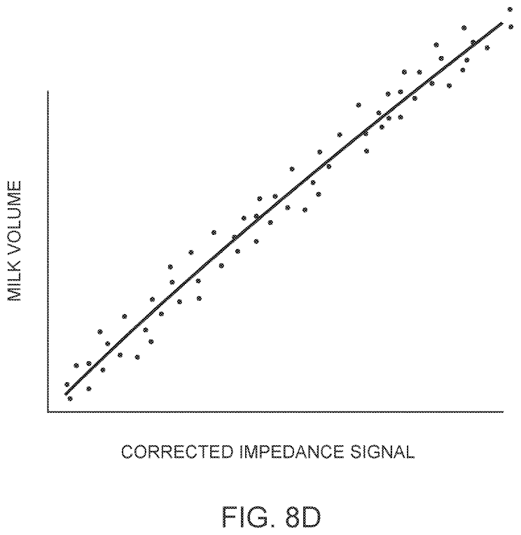

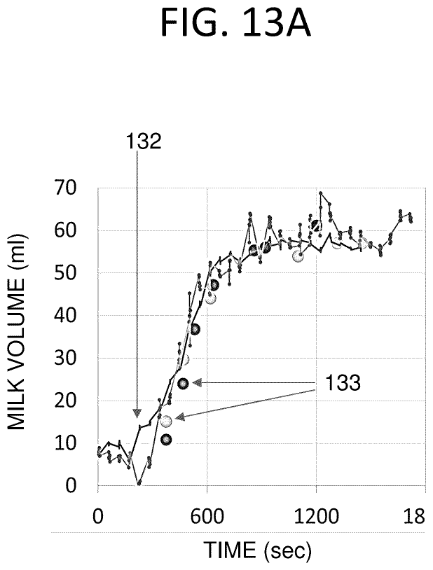

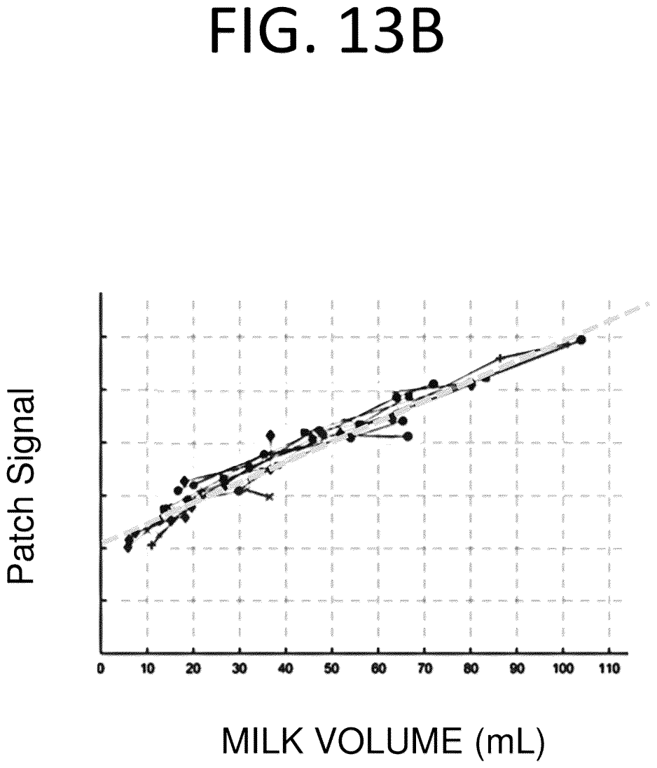

In order to convert the impedance signal into milk volume a calibration curve or calibration parameters are required. These may be obtained by measuring impedance on a given subject or a reference cohort of mothers while they express milk manually, using a breast pump to express breast milk, or breastfeed an infant. The milk volume removed from the breast is determined at periodic intervals by measuring the volume of the expressed milk using a suitable device such as a graduated cylinder or weighing a breastfeeding infant before feeding and at different time points after feeding has commenced. In this manner, a calibration curve can be construct to convert impedance parameters at given frequency, such as the real or imaginary components of impedance or the impedance amplitude or phase angle, or percentage changes in these values to milk volume. FIGS. 8D and 13B show examples of such a curve for the example of the imaginary component of impedance at a single frequency. The calibration curve thus obtained may be a simple linear curve characterized by 1 or 2 fit parameter or have a more complex shape or require more parameters.

The most common approach used in research settings has been to have each perform a calibration procedure for each mother the first time the device is used to obtain the calibration parameters and store these parameters for subsequent use. For some mothers. Different calibration parameters are required for each breast due to difference in breast size or shape between the right and left breast. While this approach is acceptable in a research setting, it is impractical for a commercial product as it requires significant effort in performing a careful calibration and may require equipment such as a breast pump, sensitive scale, or volume measuring equipment that may not be available to all mothers. On the other hand, the difficulty with using calibration data from a reference cohort is that due to anatomical differences between mothers, data collected from a reference cohort may or may not apply accurately to all mothers and it is difficult to know a priori whether such a simple universal calibration will apply to all mothers who may use the system.

The Breast Sense system solves this problem by combining the impedance sensor with a shape sensing element such as a strain gauge or camera that detects breast shape as described in detail below. The principal role of the shape sensing element is to determine parameters that characterize a given mother's breast anatomy. These parameters are then used to either directly adjust the calibration parameters obtained from a rain cohort or to use a trained machine learning model to compute suitable a calibration parameter for that subject. Furthermore, if the subject's anatomy falls outside the range for which calibration data was collected in the original cohort, the shape sensing element can signal a mother to contact a clinician or the device manufacturer for appropriate support in place of using inaccurate calibration parameters.

There are several parameters that the impedance sensor outputs can provide, including the frequency and amplitude of the drive current and voltage, the amplitude and phase of voltage detected at the sense electrodes relative to the drive current and voltage. These parameters are usually combined to report real and imaginary impedance values or resistance and capacitance at each frequency. As known to those skilled in the art, these are equivalent ways of characterizing the same data output.

Additional parameters such as resistance, capacitance, or time constant values may be derived by fitting this data to theoretical models or equivalent circuits consisting of components such as resistors, capacitors, and constant phase elements. However, it is understood by those skilled in the art that biological impedance data can usually be fit to multiple theoretical models or equivalent circuits to obtain resistor and capacitor values. Therefore, resistance and capacitance values derived from the impedance sensor output are not necessarily unique. For this discussion, the impedance sensor output will be discussed in terms of the real and imaginary components of the impedance, but it is understood by those skilled in the art that resistance, capacitance, phase, and amplitude may offer equivalent ways of describing and analyzing the same data.

The base parameter, 86, shown in FIG. 8A is the imaginary component of the impedance sensor. The impedance sensor typically operates at one or more frequencies in the range of about 0.1 to 1 MHz, such as about 1 to 100 kHz. Typically, data at between one and three frequencies in this range is collected, such as about 5 kHz, about 10 kHz, and about 20 kHz. At each of those frequencies the signal produces two numbers; a real and an imaginary value of impedance as is known to those skilled in the art.

FIG. 8A is the imaginary component of the detected impedance plotted at a particular frequency such as about 5 kHz versus time during a feeding. The data shows three distinct regions. Region 88 is the period before feeding starts, region 90 is the period during feeding, and region 92 is the period after the baby is finished feeding.

Before the feeding starts, the breast is full of milk. There is a baseline value of the imaginary component of the impedance. As the baby feeds, the imaginary component of the impedance drops to a final value at the end of feeding. During feeding, region 90, the change in the impedance signal, has a long-term change, a decline. This can be seen as the difference in the impedance signal plateaus in regions 88 and 92.

Because the impedance signal is also sensitive to the shape and deformations of the breast, there are typically undulations, or noise, associated with breathing, coughing, laughing, by the mother, the baby latching or detaching from the breast, swatting or grabbing the breast, or the mother compressing her breast to assist the baby in feeding. The magnitude of these distortions during active feeding can be small or very substantial, and as high as 2 to 3× greater the impedance change due to milk transfer out of the breast over a short period of time. These undulations are corrected by suitable filtering of the impedance signal using a band pass filter or use of the shape sensing element to correct for them in real time. Additionally, if the breast shape changes before- and after feeding, this may also affect the impedance data and may even manifest itself as slight slopes in the plateaus before and after feeding as shown in time regions 88 and 92 in FIG. 8A.

### Strain Gauge Sensor as Shape Sensing Element

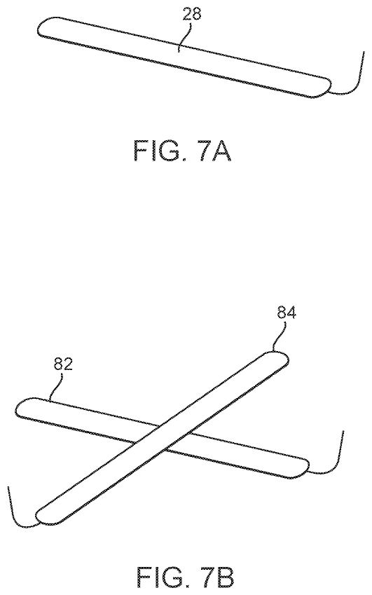

FIG. 7 shows various configurations for the optional strain gauge sensor 28. Strain gauge sensor 28 is typically a long piezoelectric sensor, whose resistance changes with how much it is bent. This is how strain gauge sensor 28 detects the curvature of the breast and movements like breathing, or deformation of the breast. The basic configuration is shown in FIG. 7A. In this case, strain gauge sensor 28 4-6 inches long and ¼ inch wide. As shown in the configuration of FIG. 7A strain gauge sensor 28 provides bending measurement in one direction. All these measurements can be varied to some degree.

Strain gauge sensor 28 can be in different patterns relative to the impedance sensor and the breast. The strain gauge may be positioned to measure breast curvature in parallel or perpendicular to the direction of the impedance sensor. When the direction of the strain gauge is parallel to the impedance sensor, as seen in FIG. 7A, the strain sensor allows correction of the impedance signal for changes to the effective distance between the impedance electrodes and to breast curvature in that direction. Alternatively, the strain sensor may be positioned at a different angle, such as 90 degrees, relative to the impedance sensor. In this configuration, it allows correction for breast curvature in a direction other than the impedance sensor. It may be desirable to have multiple strain sensors along multiple axes, such as parallel and perpendicular to the impedance sensor. As shown in FIG. 7B, two different strain gauges can be installed into the patch. While this design would make the Breast Sense Feeding Monitor device form factor larger, the design allows detection curvature and breast deformation in two dimensions.

Multiple piezoelectric strain gauges 82 and 84 can be provided in a cross pattern as shown in FIG. 7B. In other configurations, not shown in this view, piezoelectric strain gauges 82 and 84 would be provided back-to-back. This provides better measure of bend in both directions than the other direction.

The strain gauge connects to the strain sensor circuit 10 which is typically called a bridge circuit, in various manners well known to ordinary skilled artisans. By example, full bridges, half bridges, quarter bridges and other designs that translate that bending, and detecting the voltage or the resistance change that comes out of that.

The primary function of the optional strain gauge is to determine the curvature of the breast in one or more directions. Typically, the strain gauge is calibrated so that its output voltage, resistance, or capacitance reflects its curvature. When applied to the breast, the strain gauge sensor provides a signal that can be used to adjust or correct the impedance calibration parameters from a reference cohort according to well known methods for correcting impedance measurements for curvature, such as equivalent spherical or elliptical radii, normalized impedance from finite element calculation, series approximations, and other methods listed in the following references which are hereby incorporated by reference in their entirety for all purposes: (a) Smythe, W. R., Static and Dynamic Electricity, Third Edition, McGraw-Hill Book Company, New York, N.Y., 1968, Chapter IV (“Fields of Conductors”) and Chapter V (“Boundary-Value Problems”), Sections 4.06-4.09. (b) Grimnes, S. and Martinsen, Ø. G., Bioimpedance and Bioelectricity Basics, Third Edition, Academic Press, Oxford, United Kingdom, 2015, Chapter 4 (“Electrodes and Measurement Geometry”) and Chapter 6 (“Volume Conductor Theory”), which discuss the influence of surface curvature on impedance measurements and the use of spherical and locally equivalent geometries for modeling curved biological surfaces. (c) do Carmo, M. P., Differential Geometry of Curves and Surfaces, Prentice Hall, Englewood Cliffs, N.J., 1976, Chapter 4 (“The Geometry of Surfaces”), Sections 4-2 (“The Second Fundamental Form”) and 4-4 (“Mean Curvature”), which define principal radii of curvature and mean curvature and establish the local equivalence of smooth surfaces to spheres of radius equal to the inverse mean curvature. (d) Lionheart, W. R., 2004. EIT reconstruction algorithms: pitfalls, challenges and recent developments. Physiological measurement, 25(1), p. 125. (e) Yorkey, T. J., Webster, J. G. and Tompkins, W. J., 2007. Comparing reconstruction algorithms for electrical impedance tomography. IEEE Transactions on Biomedical Engineering, (11), pp. 843-852.

FIG. 8 illustrates additional ways in which the strain sensor can complement and improve the performance of the impedance sensor. FIG. 8A shows the output of an impedance sensor for a typical feeding session, including potential undulations due to breast deformation. FIG. 8B shows the signal of a strain sensor collected from the same period of time, showing the deformations that cause the undulations in FIG. 8A. In addition to using the strain sensor to adjust calibration data, the strain sensor can be used in real time to derive a normalized correction factor that is inversely proportional to the deflection detected by the strain gauge in FIG. 8B. By multiplying the impedance signal in FIG. 8A by the correction factor, the effect of breast deformations can be eliminated or minimized.

An additional use of the optional strain gauge in the Breast Sense Feeding Monitor system is to define a band of strain gauge values that are considered the acceptable range of breast deformation that allows valid impedance data to be collected. Then, in the software, impedance data collected at time points when the strain sensor output is outside this band can be rejected or averaged with a lower weight factor than data collected during periods when the strain sensor is within the acceptable band. In other words, only utilize data when the breast is not severely distorted. If the breast is distorted too much, the data collected during that time is ignored. Basically, data collected during periods of distortion is considered invalid data.

This analysis distinguishes the native shape of the breast versus when it is subject to distortion, such as when the baby presses on it abruptly. If the baby presses, then deforms the breast so that data falls outside the acceptable band, the strain gauge informs the system that something is occurring, such as, the baby is compressing the breast, the mother mom moved or was like having a coughing fit, etc.

In FIG. 8B, data band 96 is correlated with strain gauge output 94 to indicate that the shape of the breast is outside an acceptable range, and can be eliminated from the analysis. The only impedance data used are from time points where the strain sensor output is within this band 96. This eliminates a great deal of noise by eliminating the extremes where the breast is substantially deformed. As indicated above, this can be done during pre-feed time region 88, during feed time region 90, and post-feed time region 92 in FIG. 8A and FIG. 8B.

As shown in FIG. 8A, during the measurement the breast tends to make milk, even as the baby is feeding. As a result, there is oftentimes a slight slope to these pre- and post-measurements. This is observable in impedance data in region 88 and 92, seen as a slight slope to the measurement. That slope corresponds to the baseline production of milk in the breast. In the experience of some of the present inventors, this slope can be downward or upward. This depends on when the mother's milk starts to be produced. This can be subtracted from the baseline, because typically the milk production is very slow. Being able to correct for the baseline production of milk is a unique capability of the Breast Sense Feeding Monitor system.

### Detection of Suck Patterns

FIG. 9 demonstrates a further aspect of the Breast Sense Patch 34 that allows detection of additional feeding parameters such as sucks and swallows in addition to milk volume. If the Breast Sense Patch is close enough to the latch area on the breast, the impedance sensor output signal is also sensitive to distortions of the breast tissue causes by the sucking and swallowing movements in the infant's mouth. These distortions can have a characteristic pattern, as shown in FIG. 9B. The signal or y-axis in FIG. 9B is the real component of the impedance signal in ohms. The x-axis is time in seconds. The characteristic pattern for sucks and swallows consists of about 1 to 5 small amplitude waves (sucks) followed by a deep wave (swallow). The detecting sensor must collect about 5 to 10 data points a second in order to detect sucks and swallows. In most infants, sucks occur approximately once every 0.8 to 1 second. Swallows occur approximately once every 1 to 5 seconds. In order to collect about 5 to 10 data points a second, the impedance sensor would run at a single frequency so that data at that frequency is collected and averaged every 100 msec or sooner. By comparison, for the detection of milk intake, the impedance sensor should be run at 2 or more frequencies, and data for each frequency should be average over 200 msec to 1 sec. This means that in multi-frequency mode, time points will be 1 to 5 seconds apart.

The inventors have discovered that what works best is to alternate between running the impedance measurement at a single frequency with running the impedance at multiple frequencies. FIG. 9 shows the frequency used for impedance measurement. By example, a single frequency, for example, 10 kHz, is run very quickly, so back to back 10 kHz measurements every 100 ms. This is the single frequency fast region 98. Then every 30 s-2 min two other frequencies are run, as in multi-frequency region 100. So for example, this could be done at 20 kHz and 5 kHz.

During each of these, the Breast Sense Feeding Monitor system is constantly going back and forth between a single frequency region and multi-frequencies. Then a single frequency is run again, followed by multiple frequencies. The multiple frequencies are seen as the three parallel lines in the table. Single frequencies are one line.

Alternatively, multiple frequencies can be run during the pre-feed 88 and post-feed 92 time regions while a single frequency is run during the active feeding region 90.

The reason a single frequency is run is that when during a single frequency reading, running a single frequency can collect data very quickly, always at the same frequency, it is possible, for the first time, to detect the baby's sucks and swallows. This innovative functionality is shown in FIG. 9B. FIG. 9A shows the operation of the Breast Sense Feeding Monitor system at different frequencies.

FIG. 9B shows showing detection of suck during the single frequency part of the measurement. During the single frequency part of the measurement, the impedance signal has very fine waves corresponding to sucks 102. These can be small rapid undulations, by example, that are superimposed on the larger signal. Then there are some dips that are swallows in between. After that the typical baby then goes into a pattern on suck suck suck, and gulp, suck suck suck, and gulp. This is shown as swallows 104, and sucks 102.

The rapid, single frequency mode of operation allows detection of sucks and swallows, which has two benefits. One is, it actually allows a mother to assess the quality of the infant feeding to help to optimize latch or how the baby is held. A baby that is well-latched will have a pattern of consecutive strings of sucks and swallows (for example, suck suck suck suck, swallow, suck suck suck suck suck, swallow . . . ) with few breaks. The baby that does not have a good latch or that is struggling with feeding, e.g., due to being premature or having neurological or motor problems, will have irregular patterns of sucks and swallows, interspersed with periods where the baby detaches from the breast, cries, or takes a rest.

The second benefit of this fast measurement is that the sucks and swallows can be counted. Assuming a certain volume is swallowed, or is pulled for a typical suck, this is useful to error-check the standard impedance measurement. The result is more robust data. This provides a second way to calculate milk transfer. At least during that region, the average of the two can be taken. Alternatively, they can be combined in different ways.

For example, for infants younger than 2 or 3 days, milk production has not commenced or is too low to detect reliably through the standard impedance measurement (FIG. 8A). During this time, infants feed on the thick viscous fluid produced by the breast known as colostrum. Infants typically lose weight during this period, resulting in substantial anxiety among mothers as they wait for their milk to “come in”. During this period, it is possible to provide a measure of the progression towards onset of milk production (lactogenesis) by measuring and tracking the suck-to-swallow ratio and the number of suck-swallow bursts per minute. Providing these mothers with a measure of progression can substantially alleviate anxiety. It can also quickly identify mother-infant pairs where there may be a delay in lactogenesis and additional interventions such as supplementation or the use of a breast pump may be appropriate.

Alternative configurations of the Breast Sense Patch are possible that allow detection of the suck and swallow data with greater accuracy.

For example, in the Breast Sense patch configuration of FIG. 4A, one pair of electrodes may be used primarily for detecting sucks and swallows and a different pair for detecting milk volume. The different pairs may be connected to a single impedance sense circuit that switch between different pairs. Alternatively, they may have dedicated impedance sense circuits that allow simultaneous measurement of both pairs.

Sucks and swallows may also be detected by the strain sensor located inside the Breast Sense Patch, in place of or in addition to the impedance sensor. The strain sensor may superior better sensitivity. Both sensors may be used to detect sucks and swallows in order to cross check each other and eliminate artifacts.

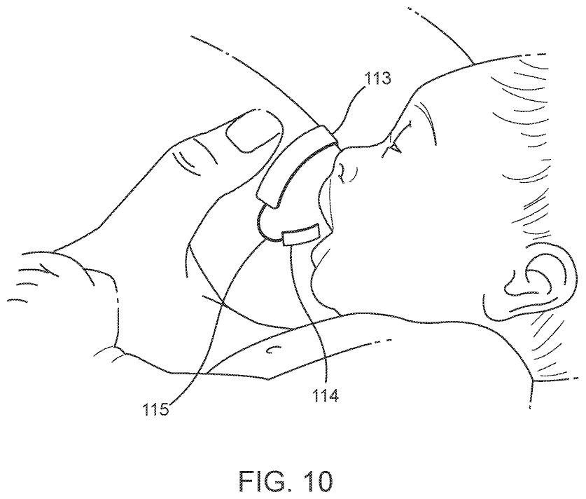

Alternatively, in FIG. 10, the sucks and swallows may be detected using 1 or more additional impedance sense electrodes or a dedicated strain sensor positioned inside or very close to the area where the baby latches onto the breast and connected to the Breast Sense patch via wires. While this location may not be ideal for detecting milk volume, it can provide higher sensitivity for detecting suck and swallow motions.

Alternatively, a second patch, called the Baby Patch 116, may be placed on the infant's throat, neck, or chin area that is specifically used for detecting sucks and swallows. This is shown in FIG. 11A. The Baby Patch may contain either an impedance sensor or strain sensor or both. It may attach to the baby's throat, chin, or cheek area, preferably underneath the chin where maximal displacements can be detected due to sucking and swallowing motions. It may connect wirelessly or via wires to the Breast Sense Patch 114. In some embodiments, the Baby Patch 116 sends its digital signal wirelessly to the mobile phone 38. In other embodiments the Baby Patch may send its signal wirelessly to the Breast Sense Patch 114 where the signals from the two patches can be combined to avoid latency issues that may arise if the signals are sent separately to a mobile phone that is running multiple software programs at the same time.

The Breast Sense Patch and the Baby Patch may be held in place using any suitable mechanism, including but not limited to the adhesive used in gel electrodes or other suitable hypoallergenic skin adhesive suitable for contact for the duration of multiple feedings.

The following is offered by way of illustration and not byway of limitation.

### EXAMPLES

Example 1—FIG. 8C shows data from the Breast Sense Patch using impedance and strain sensor data. The impedance sensor output 84 was the imaginary component of the impedance at 50 kHz measured with a 6 inch Breast Sense Patch using gel electrodes (3M 2228 gel electrodes) and a piezoelectric strain sensor (4.5″ long piezoelectric, with a 2×10 kohm half bridge strain sensor circuit). The Breast Sense Patch was worn on the breast as depicted in FIG. 2A, at distance of 6 cm from the nipple. Impedance data was collected with a drive current of 500 uA at 5, 10, 20, 50, and 80 kHz during breastfeeding.

Line 84 was the imaginary component of the detected impedance at 50 kHz. This corresponds to the right axis in FIG. 8C. The strain sensor output 94 corresponds to the left axis and has arbitrary units. An upward deflection in the strain sensor indicates compression of the breast. At approximately 330 seconds from the start of feeding, the infant's movement caused a significant distortion of the breast from 2000 to 400 sensor bits.

This resulted in a corresponding step increase in the impedance signal from 2.2 ohm to 2.75 ohm, caused entirely by the deformation of the breast and does not indicate milk transfer. Another large change in breast shape occurs at approximately 450 seconds and is detected by deformation is detected by the strain sensor output 94. This resulted in a large downward change in the impedance curve 84.

After 500 seconds, the breast returned to an un-deformed state and the impedance data 84 is much less noisy. Using the strain sensor data to identify the regions of significant breast distortion and correct for the distortion allowed the impedance data to be corrected to obtain curve 99. The improvement in noise going from curve 84 to curve 99 could not be achieved simply by averaging or using the impedance data alone. Curve 99 was significantly less noisy and represents changes in impedance related to milk volume in the breast independent of breast deformation.

Example 2—FIG. 11B shows a typical real time output of milk that was transferred, in mL of milk versus time, based on the Breast Sense Patch where the impedance signal was corrected with the strain gauge signal.

Example 3—FIG. 11C is typical output from a Baby Patch, as would be located on a feeding infant's throat area. In this example, and using a strain gauge sensor output whose deflection indicate sucks 122 and swallows 120.

### Camera and Photogrammetry as Shape Sensing Element

An alternative embodiment of the Breast Sense system uses a camera, optical, or LIDAR sensor as the shape sensing element in place of a strain gauge sensor built into the patch. The strain gauge sensor adds cost and bulk to the Breast Sense Patch. It requires calibration at either factory or point of use which also adds complexity and cost to device production and testing. Conversely, most mothers possess mobile phones with built in cameras that can be used with relatively little effort to measure breast shape and additional such as patch location and breast texture that may provide the same or better information than a strain gauge for this application.

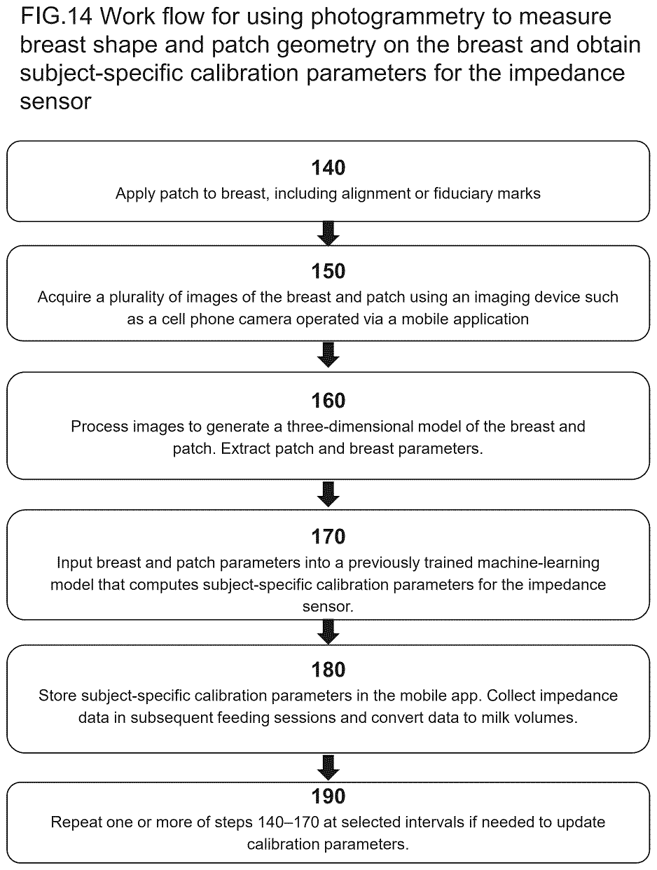

This can be accomplished by collecting images with a mobile phone or portable camera and combining them into a 3D model of the breast using photogrammetry. FIG. 14 shows a flowchart depicting the steps involved in combining photogrammetry with impedance measurements. The broad steps consist of (1) image collection (FIGS. 14, 140 and 150), (2) 3D model construction and extraction of parameters that describe the breast and patch dimensions and key features (160), and (3) insertion of the extracted into a machine learning model (170) such as a neural network to obtain subject-specific calibration parameters that (180) convert impedance measurements to milk volume for each subject.

### Image Collection

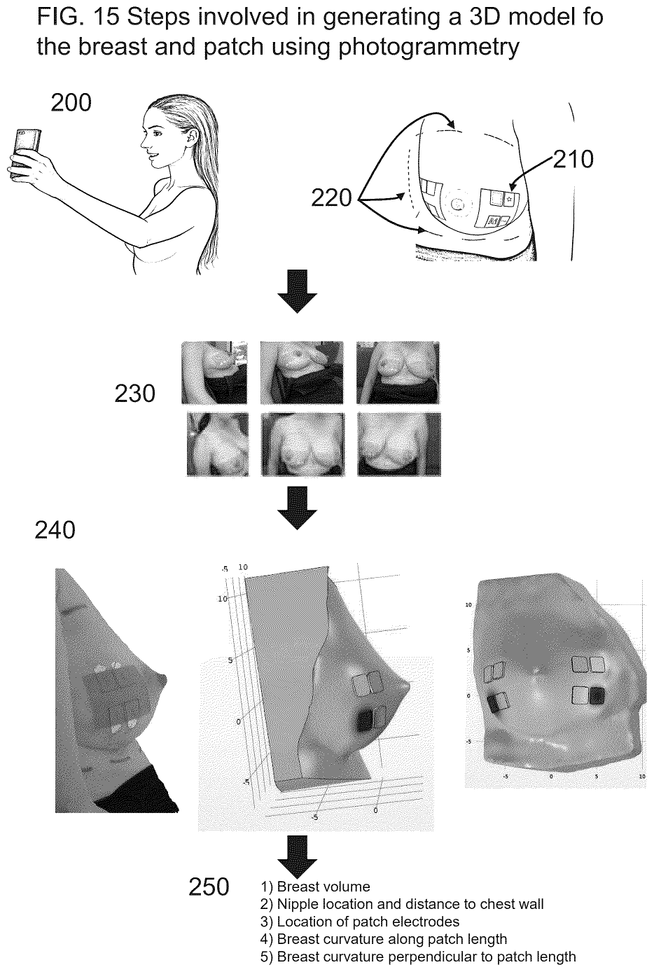

FIG. 15 shows the steps involved in collecting images with a camera (200) to construct a 3D model via photogrammetry. After application of the patch (210) to the breast, the subject uses a camera such as the one on their mobile phone to capture photographs of the patch as applied to the breast from different angles. Because the subject may have difficulty manipulating the camera without causing deformation of the breast, it may be advantageous to have an assistant such as a family member or a clinician hold and move the camera.

Alternatively, the camera may be attached to an arm (similar to a selfie stick) that ensures consistent camera distance and can be moved by the subject with minimal effort. Alternatively, the camera may be on a robotic arm that sweeps in a predefined trajectory without any effort by the subject. Alternatively, the camera may be stationary, such as fixed to a tripod, and the subject may turn her body or be seated be on a rotating seat or turntable that allows images to be collected from different angles. Alternatively, images may be taken by multiple cameras from different angles.

Depending on the shape of the patch, for example whether the patch distorts the breast shape, it may be advantageous to collect images of the breast both with and without the patch applied or with and without devices such as a breast shield or breast pump applied to the breast.

The photos may be collected by taking individual photos, a burst of photos or video as the camera is swept along the desired trajectory to capture images of all sides of the breast, for example top, bottom, front, and side views. The mobile app may provide instructions to ensure a sufficient number of photos are collected with adequate focus and lighting for processing. Instructions may be a combination of text, video, photo, or voice prompts.

In practice, the inventors have found that a series of 3 sweeps, with 5-10 images collected with the camera pointing down at the breast, 5-10 images collected with the camera swept at nipple level, and 5-10 images collected with her camera pointing up at the underside of the breast are sufficient for constructing a model without good resolution without requiring excessive time or effort by the subject. A sampling of such photos is shown in FIG. 15 (230). This ensures that the breast is imaged from all angles with sufficient overlap between the photos to construct a 3D model with the relevant features of the breast and patch features, even if some photos were to have problems such as poor focus. In a commercial system, the ultimate number of photos required may be higher or lower depending on (a) the imaging hardware, (b) the software algorithm's accuracy and sensitivity, (c) the level of effort tolerated by the subject (d) the use of alignment or fiduciary marks and features.

To ensure consistent lighting and avoid shadows that could be misinterpreted as anatomical features during image processing, the camera may be equipped with a suitable light, such as a ring light. Alternatively, the subject may be required to sit in a well-lit room with specific lighting requirements. The software may also adjust images to improve lighting and focus.

In certain embodiments, it is advantageous to have fiducial markers, calibration markings, or reference objects on the patch, breast, or surrounding garments. These markings may be printed during manufacturing onto the patch itself or applied to the breast at the point of use, for example using pre-printed stickers, or a suitable marker, stamp, or temporary tattoo.

The inventors have found fiduciary marks that indicate the location of the sternum (220), the inframammary fold where the bottom of the breast attaches to the torso, and the approximately boundary of the breast region (i.e. the area outside of which the photogrammetry algorithm can ignore any pixels) to be advantageous. Furthermore, its is advantageous if at least one fiducial mark that provides a known length scale is present in each photo to allow rapid construction and scaling of the model. The mark can be a line or marking of known length similar to the scale on a map that is printed on the patch or applied to the breast using a sticker or stamp. It may also be letters, numbers, or shapes such as a star shape as shown in FIG. 15 210. The outline of the patch itself may serve as a fiduciary marking for scaling provided it is of fixed and known dimensions and not excessively distorted after application to the breast.

Fiduciary markings that are captured in images, may serve several additional functions to the ones above, including but not limited to (a) providing a means of calibrating distances in the photos so that the 3D model can be scaled correctly to breast dimensions and patch location accurately, (b) providing a landmark or registration mark that allows images to be combined more accurately or efficiently during processing, (c) assisting the camera's autofocus mechanism, (d) enabling the software to check in real time if an image is out of focus or the lighting is inadequate or inconsistent for processing so that the image may be retaken, (e) indicating the location of key anatomical features that may be difficult to distinguish in the photos if unmarked, such as the sternum, the inframammary fold where the bottom of the breast attaches to the torso, the top and side boundaries of the breast's soft tissue, the location of the areola, or the location of any scars or other anatomical features that may affect either the 3D model construction or the impedance, (f) ensuring that the sweeping motion photos have captured the full extent of the breast, i.e., that one side of the breast is not obscured, (g) making the image processing algorithms faster, more efficient, or more accurate, by providing readily recognizable features (h) minimizing the number of photos required, including potentially a single image and (i) providing unique identifiers for a subject such as a serial number, bar code, or QR code to allow rapid location of subject data in a cloud-based database.

Once images are collected, the mobile phone app may review the images in real time or at the end of the sweeps and alert the subject or assistant if images are adequate or if additional images need to be taken or retaken. The checks include verifying that there are a sufficient number of in-focus images, that the entire breast has been imaged, and that the lighting between images is consistent for processing.

In addition to providing input data for building a 3D model of the breast, the images collected during this step may be stored in a database. After collection of data from a sufficient number of subjects, these images may be used for comparison to future subjects and provide an indication or predictive diagnosis of breast anatomies or appearances indicative of potential breastfeeding or other health issues. Examples include specific sizes and shape of the nipple, skin conditions or scarring, or breast size and shape in conjunction with other factors such as patient history or biometrics.

While the preferred embodiment described above uses an optical camera on a mobile phone or portable camera, other imaging modalities such as infrared light and LiDAR can also be used as the shape sensing element.

### 3D Model Construction

Once images 230 have been obtained, they are used to construct a 3D model of the breast 240 using established photogrammetry algorithms. These algorithms are in principle similar to those used in photography to stitch a series of photos together and generate a panorama image.

The steps in processing the photos and constructing a three-dimensional model of the breast are as follows: (1) detection of image features and identification of corresponding features across multiple views using scale-invariant feature extraction methods, such as the Scale-Invariant Feature Transform (SIFT), to establish reliable feature correspondences; (2) estimation of camera parameters and sparse three-dimensional structure using a Structure-from-Motion (SfM) framework, in which the camera pose—comprising camera position (translation) and orientation (rotation)—and, in some implementations, intrinsic calibration parameters including focal length, principal point, and lens distortion coefficients are jointly estimated along with a sparse representation of the breast surface; and (3) dense three-dimensional reconstruction using a Multi-View Stereo (MVS) process that computes per-view depth information and fuses it into a dense point cloud. Together, these methods constitute a widely adopted photogrammetric pipeline that provides robust and accurate three-dimensional reconstruction from unordered two-dimensional images.

Although SIFT, SfM, and MVS are preferred due to their robustness and widespread adoption, alternative algorithms may be employed at one or more stages of the reconstruction pipeline. Feature detection and matching may be performed using binary feature descriptors (e.g., Oriented FAST and Rotated BRIEF (ORB)), affine-invariant features (e.g., Speeded-Up Robust Features (SURF)), or learned feature representations produced by trained machine-learning models (e.g., SuperPoint). Sparse reconstruction and camera parameter estimation may alternatively be achieved using visual simultaneous localization and mapping (SLAM) or direct methods, such as ORB-SLAM or Direct Sparse Odometry (DSO), which jointly estimate camera motion and scene structure. Dense reconstruction may be performed using depth-map fusion techniques, volumetric reconstruction methods (e.g., truncated signed distance function (TSDF) fusion), or learning-based multi-view depth estimation models (e.g., Multi-View Stereo Networks (MVSNet)). The selection among these alternatives may be guided by application-specific considerations including imaging conditions, computational resources, and reconstruction accuracy requirements.

Image processing and 3D model construction may be performed by software on the mobile phone or the images may be uploaded to a cloud server for processing. For patient privacy reasons, the preferred approach is to construct and store the model on the subject's mobile phone rather than a centralized server. However, processing images on a mobile phone has the disadvantage of slow speed due to reduced processing power and memory compared to a cloud server and may result in excessive battery usage. The preferred approach must be selected based on these considerations and may involve a combination of the two or evolve as greater computational power becomes available in future generations of mobile devices.

### Machine Learning Model

Once collected and processed, the 2D images and 3D model are used as inputs to a neural network or other machine learning model trained to provide the most accurate and appropriate calibration curve for each subject or each breast. The latter is important because for most women, the right and left breasts are not identical in size. They may also have differences in features such as glandular tissue content and distribution, skin resistance, scars or tattoos, or patch placement. Therefore, in practical applications, it is likely that each breast will require a unique set of calibration parameters.

The neural network model is developed, trained, and validated using photogrammetry and impedance calibration curves obtained from a reference cohort of subjects. After training, the model may be maintained and used in the cloud or downloaded to the user's mobile device. It may be further updated over time as new data become available, for example as patients with new demographics or medical conditions use the system.

The model uses input parameters to either “look up” the closest available calibration curve (discrete calibration) or to interpolate a calibration curve suitable for each subject (continuous calibration). A continuous calibration provides greater accuracy at the expense of greater computational cost and memory usage, including greater battery energy consumption if implemented on a mobile app.

An example of possible input parameters to the model is given in FIG. 15 240. This list is provided as an example and is not exhaustive. In practical application, the inputs to the machine learning model may include a combination of a variety of parameters and formats, including:

(1) Tabular scalar or vector values representing the breast and extracted from the 3D model, such as computed breast elliptical axes (Rx, Ry, Rz), breast volume, surface area, centroid or center-of-mass coordinates, breast projection (distance from chest wall to the most anterior point), distance from the chest wall to the nipple, base width and height, nipple position and orientation, surface curvature metrics such as mean and Gaussian curvature, symmetry metrics comparing left and right breasts, shape descriptors such as compactness and sphericity, and distance maps.

(2) Tabular scalar or vector data representing the size and location of impedance electrodes or patch, including but not limited to electrode size, electrode coordinates, patch curvature, inter-electrode shortest distance, and patch distance and angle relative to the chest wall and nipple. While the preferred embodiment is a path of pre-defined length, photogrammetry allows patches of different lengths to be used, for example by applying individual electrodes or sets of electrode to the breast without a predefined spacing to maximize comfort. As long as the calibration data set includes electrodes of the same spacing, photogrammetry allows a patch with individually placed electrodes to be used.

(3) Impedance data from the impedance sensing electrodes, such as the initial or final values of impedance between two or more electrodes, current flow, or voltages between various pairs of electrodes. The impedance data may also be obtained from multiple electrodes such as those in FIG. 4B. It may also be in the form of an impedance map generated via electrical impedance tomography performed by a patch such as that shown in FIG. 4G. In this instance, the location of glandular tissue inside the breast may be extracted from the impedance map and input into he model for both training purposes and to obtain accurate calibration data.

(4) Surface features, including but not limited to color and texture of the breast skin, and the shape or location of surface features such as the nipple, areola, visible blood vessels, or scar tissue. For example, darker skin tone is known to correlate with higher skin resistance. The location of the nipple or areola may correlate with the distribution of milk-containing glandular tissue inside the breast. While it is difficult to establish such correlations a priori (i.e., before collecting data from a wide range of subjects), neural networks are well suited to processing such data and identifying predictive features.

(5) Biometric data provided by the subject and entered into the app, including but not limited to subject age, height, weight, BMI, race, skin tone, and medical conditions or history.

(6) In place of parameters extracted from the 3D model or 2D images, the input to the neural network may also include images or graphical representations of the data. This includes the 2D images themselves, a mesh representation of the breast shape, or 2D curves representing cross sections of the breast shape.

(7) Synthetic data: it is also possible to supplement calibration data from human subjects with synthetic data. An example of this is interpolating between the original calibration data sets using finite element simulations or correcting for small deviations in parameters such patch length, location, or electrode area from the original calibration data set. Rather than collecting additional data with human subjects for every possible case, simulations may be used to fill gaps between calibration data bins or sets.

The neural-network architectures suitable for the model include multilayer perceptron (MLP), convolutional neural networks (CNN), graph neural networks (GNN), and models with an autoencoder and regression head for latent-space learning. The choice among these architectures depends on the input information and how it is represented. For example, an MLP is preferred for tabular input data, a CNN for spatially aligned surface or thickness maps, a GNN for mesh- or graph-based surface representations, and an autoencoder-regression framework to learn compact latent shape descriptors and reduce the dimensionality of the input parameter space prior to prediction.

Compared to a strain sensor, the main advantage of photogrammetry for calculating breast shape and obtaining subject- or breast-specific impedance calibration curves is a reduction in hardware complexity and cost associated with the strain sensor. Specifically, components 10 and 28 in FIG. 1 are eliminated, resulting in lower patch manufacturing and design cost and a potentially smaller, lighter, and more comfortable patch. Furthermore, photogrammetry uses hardware that is readily available—namely a cell phone camera—to obtain a complete set of dimensions and shape data for the breast, potentially providing higher accuracy than is possible with an individual hardwired strain sensor.

### Model Training

To train the machine learning model, data must be collected from a large number of subjects with a range of breast sizes and shapes, such as at least 50, and preferably at least 100 subjects. Depending on the parameters used as input to the machine learning model, it may be desirable to include subjects that cover ranges of additional characteristics such as age, height, weight, BMI, race, skin tone, or specific medical conditions.

The training data is generated by collecting (a) photogrammetry data on each subject and (b) measuring a calibration curve of impedance vs milk volume for each subject. The method is to have the subject wear a Breast Sense Patch while using a breast pump is express milk from the breast. The milk is collected at periodic intervals to measure the total milk expressed up to that point and generate a calibration curve of impedance vs milk volume for each subject and breast. The version of the Breast Sense Patch used in this step would be one without a strain gauge and potentially designed to be easily applied and aligned when using a breast pump. The calibration data can also be generated by other means, such as manually expressing milk from the breast and measuring its volume or weighing a breast feeding baby ad regular intervals to determine the volume of milk intake. However, using a breast pump is the preferred approach for generating the training data as it generates the most data with the least effort for the mother.

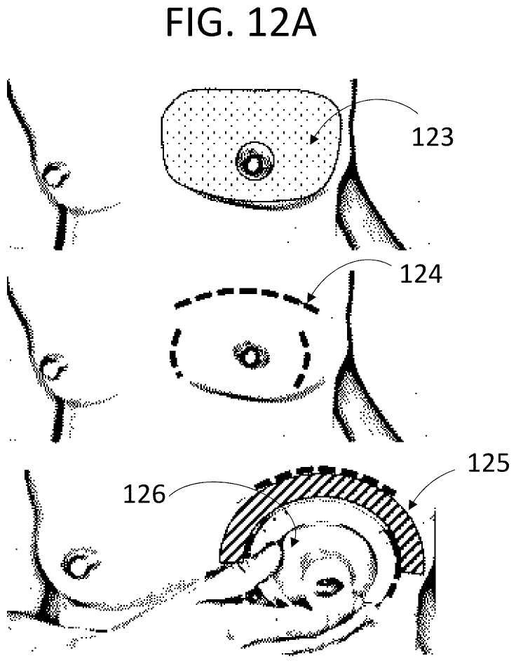

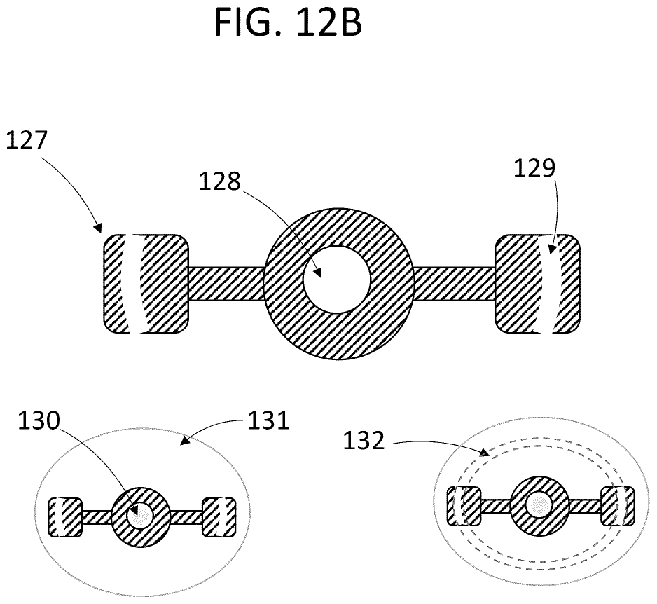

Example 4—FIG. 12A shows an example of such a patch applied to the breast to generate training or calibration data. In this example, markings 124 are applied to the breast using a temporary tattoo 123 or marker. The markings guide the placement of the patch when the mother is using a breast pump or breastfeeding a baby and ensure consistent placement in both situations. The markings indicate the position and orientation of the patch 125, breast pump flange 126, and nipple relative to each other. An alternative approach is a patch with design features that provide alignment to both breast and breast pump flange. This is shown in FIG. 12B where patch 127 includes a circular section 128 designed to align it to be aligned to the nipple 130 for breastfeeding and a groove, alignment mark, or attachment feature 129 that allows it to be aligned to a breast pump flange 132. Depending on the design, the patch may be attached to the breast or after application of the breast pump flange or it may be attached to the breast pump flange and applied in one step. Either approach is acceptable as long as the patch is consistently positioned in the same location relative to the flange and breast.

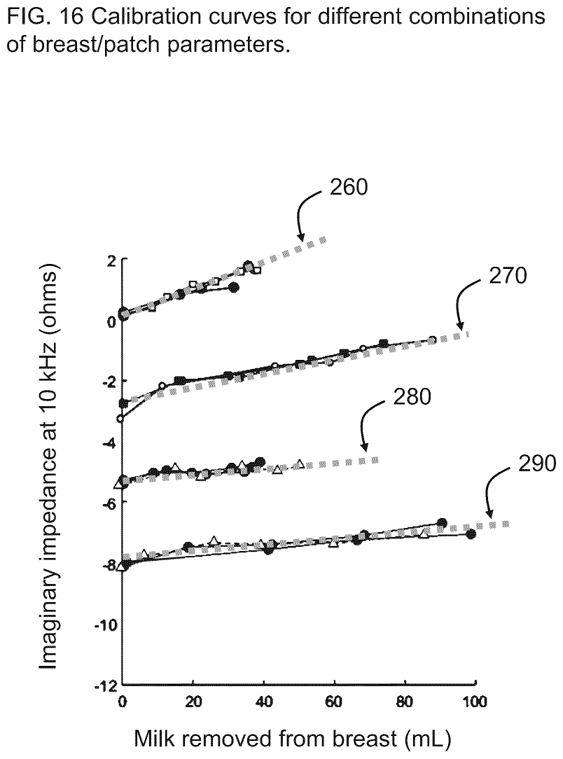

Example 5—FIG. 13A shows an example of typical data collected with the configuration described in FIG. 12A. Lines 133 were data collected by the patch in different feeding sessions. Circular points 132 were milk volumes collected using a breast pump. FIG. 13B shows typical calibration data obtained with this method for one group of subjects. Subjects with different breast anatomies are expected to have calibration curves that are similar or identical but different from subjects with different anatomy. FIG. 16 shows a simplified illustration of this concept, showing different calibration curves (260-290). Each curve is the average of calibration data from multiple subjects with similar breast characteristics and is characterized by 2 parameters, a slope and an intercept.

For example, subjects with breast volumes below 2000 mL may have calibration curve 260, those with breast volumes between 2000 and 3000 mL may have calibration curve 270, those with breast volumes between 3000 and 4000 mL may have calibration curve 280, and so on.

FIG. 16 is included merely for illustration purposes. The inventors have found that a single input parameter such as breast volume is insufficient to reliably and unambiguously assign a calibration curve to a given subject. Instead, a useful model must utilize a combination of input parameters such as those shown in FIG. 15 240 and discussed above. The strength of the machine learning approach to this problem is that multiple parameters can be used to group or bin subjects and the model can be trained and updated to provide highly accurate calibration parameters for different subjects.

Another embodiment of the Breast Sense monitor would use a LIDAR sensor to determine breast shape in place of or in addition to photogrammetry. Light detection and ranging (LiDAR) can be used to determine the size and shape of a woman's breast by emitting pulses of laser light toward the breast surface and measuring the time-of-flight of the reflected signals to generate a dense three-dimensional point cloud representing the external anatomy. LIDAR sensors are standard in some newer models of cell phones such as the Apple iPhone 16. By moving the cell phone to acquire LiDAR data from multiple angles, the individual point clouds may be aligned using point-cloud registration algorithms such as Iterative Closest Point (ICP) or generalized ICP, followed by denoising and smoothing using statistical outlier removal or moving least-squares filtering. The breast region can then be modeled using region-growing methods or model-based approaches such as RANSAC plane removal. A continuous 3D surface model may be constructed from the processed point cloud using surface-reconstruction algorithms such as Poisson surface reconstruction or ball-pivoting, and subsequently refined with mesh-smoothing and decimation techniques. The resulting three-dimensional mesh enables quantitative assessment of breast geometry, including volume, surface area, curvature, and spatial landmarks, providing a high-fidelity, non-contact representation suitable for medical analysis or integration with other imaging modalities.

The advantage of LIDAR over optical imaging and photogrammetry is (a) direct measurement of depth instead of processing images. This can result in faster and less computationally intensive generation of the 3D model. (b) direct measurement of certain dimensions such the distance between nipple and chest wall without the need for any 3D model construction and (c) reduced sensitivity or need to control ambient light compared to photogrammetry. The main disadvantage of this type of shape sensing element is that it provides no information on surface texture of the breast tissue such as scar tissue or any features or marking that do not have a raised profile.

Other embodiments of the Breast Sense system may use technologies closely related to LIDAR and photogrammetry to obtain breast shape. In one embodiment, a mobile phone with a Time-of-Flight camera may acquire three-dimensional geometric information of a breast by actively illuminating an external surface of the breast and measuring depth at a plurality of surface locations. The sensor emits modulated infrared radiation and determines a phase shift or propagation delay of reflected radiation on a per-pixel basis to generate depth data. The depth data may be processed to generate a three-dimensional representation of the breast surface that characterizes breast size, contour, and curvature.

In another embodiment, a mobile device uses a Structured Light Sensor projects a predefined light pattern onto a surface of the breast and captures one or more images of the projected pattern using an image sensor. The captured images are analyzed to determine spatial deformation of the pattern relative to a reference geometry, and depth values are computed for corresponding surface points using triangulation techniques. The computed depth values are assembled into a three-dimensional point cloud and converted into a continuous surface model using surface-reconstruction algorithms, thereby producing a non-contact, three-dimensional model of breast shape suitable for quantitative analysis or integration with additional measurement modalities.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in anyway by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail byway of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A breast sense feeding monitor system comprising:
a) a wearable device including an impedance sensing circuit with two or more impedance electrodes,
b) a shape sensing element, and
c) a user interface device which receives and displays the sensor data and controls the system.

2. The system of claim 1, wherein said shape sensing element is a strain gauge sensor, a cell phone, a portable camera, a LIDAR sensor, a Time of flight camera, and/or a Structured Light Sensor.

3. The system of claim 2, wherein the output of said shape sensing element is used to compute a correction factor for adjusting the data from said impedance sensor.

4. The system of claim 2, wherein the output of said shape sensing element is used to generate a complete or partial 3D model of a breast of a subject.

5. The system of claim 2, wherein the output of said shape sensing element is images processed with a photogrammetry algorithm to generate a full or partial 3d model of said breast.

6. The system of claim 2, wherein the output of said shape sensing element is used as one of the inputs into a machine learning model that calculates a correction factor to adjust the impedance data for each of said subject or breast.

7. The system of claim 5, wherein said machine learning model is trained using impedance, shape sensing element, demographic, and/or anatomical data collected from a reference cohort of subjects.

8. The system of claim 2, wherein said image sensing element is a cell phone or portable camera that collects a single image or plurality of images from multiple angles.

9. The system of claim 7, wherein said data input to said machine learning model is one of: tabular or vector data characteristic of breast shape, breast anatomy, patient biometric data, patch size and location; synthetic data; impedance map obtained from impedance tomography; images characteristic of the breast surface; images or graphical representations of breast or patch shape, and/or synthetic data.

10. The system of claim 6, wherein said machine learning model is any one of a multilayer perceptron (MLP), a neural network such as a convolutional neural network (CNN), a graph neural networks (GNN), or an autoencoder with regression head for latent-space learning.

11. The system of claim 1, where said wearable device includes at least one of a microprocessor, a memory chip, an A/D converter, a power source, and a wireless data transmitter.

12. The system of claim 2, wherein two or more shape sensing elements are used.

13. The system of claim 1, wherein said wearable device is a flexible patch applied to said breast.

14. The system of claim 1, wherein said wearable device includes one more impedance electrodes or strain gauge sensor positioned near the infant latch area to detect suck patterns with high accuracy.

15. The system of claim 4, wherein the output of said shape sensing element is processed using software on said patient's mobile device.

16. The system of claim 4, wherein the output of said shape sensing element is uploaded to a cloud server and processed on said server.

17. The system of claim 6, wherein said machine learning model is trained on a cloud server and downloaded to a patient mobile device prior to being used to generate calibration parameters.

18. The system of claim 6, wherein said machine learning model is trained and resides on a cloud server.
